SEQUENCE LISTING (59 sequences)

SEQ ID NO: 1 - protein, 445 aa, Zea mays
MAGAEEIVLQPIKEISGTVKLPGSKSLSNRILLLAALSEGTTVVDNLLNSEDVHYMLGALRTLGLSVEADKAAKRAVVVGCGGKFPVEDAKEEVQLFLGNAGTAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 2 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDAKEEVQLFLGNAGTAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 3 - protein, 445 aa, Zea mays
MRKWEEIVLQPIKEISGTVKLPGSKSLSNRILLLAALSEGTTVVDNLLNSEDVGYMLGALRTLGLSVEHDEAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRAEHSDSWDRFYIKGGQKYESPKNAYVEGDASSASYFLAGAAITGGTVTVEGCGTTSLQGDVKFAEVLEMMGAKVTWTETSVTVTGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 4 - protein, 445 aa, Zea mays
MRKWEEIVLQPIKEISGTVKLPGSKSLSNRILLLAALAEGTTVVDNLLNSEDVLYMLGALRTLGLSVEHDKAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVKAEHSDSWDRFYIKGGQKYLSPKNAYVEGDASSASYFLAGAAITGGTVTVEGCGTTSLQGDVKFAEVLEMMGAKVTWTETSVTVTGPPREPFGRKHLKAIDVNMNKMPDAAMTLAVVALFADGPTAIRDVASWRVKETERMVAICTELTKLGASVEEGPDYCIITPPPKLNVTAIDTYGDHRMAMAFSLAACAEVPVTIRDPGCTRKTFPDYFDVLSTFVKN

SEQ ID NO: 5 - protein, 445 aa, Zea mays
MRKWEEIVLQPIKEISGTVKLPGSKSLSNRILLLAALSEGTTVVDNLLNSEDVHYMLGALRTLGLSVEHDKAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVKAEHSDSWDRFYIKGGQKYESPKNAYVEGDASSASYFLAGAAITGGTVTVEGCGTTSLQGDVKFAEVLEMMGAKVTWTETSVTVTGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACAEVPVTIRDPGCTRKTFPDYFDVLSTFVKN

SEQ ID NO: 6 - protein, 445 aa, Zea mays
MRKWEEIVLQPIKEISGTVKLPGSKSLSNRILLLAALSEGTTVVDNLLNSEDVHYMLGALRTLGLSVEHDKAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVKAEHSDSWDRFYIKGGQKYESPKNAYVEGDASSASYFLAGAAITGGTVTVEGCGTTSLQGDVKFAEVLEMMGAKVTWTETSVTVTGPPREPFGRKHLKAIDVNMNKMPDAAMTLAVVALFADGPTAIRDVGSWRVKETERMVAICTELTKLGASVEEGPDYCIITPPPKLNVTAIDTYGDHRMAMAFSLAACAEVPVTIRDPGCTRKTFPDYFDVLSTFVKN

SEQ ID NO: 7 - protein, 445 aa, Zea mays
MRKWEEIVLQPIKEISGTVKLPGSKSLSNRILLLAALSEGTTVVDNLLNSEDVLYMLGALRTLGLSVEHDKAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVKAEHSDSWDRFYIKGGQKYESPKNAYVEGDASSASYFLAGAAITGGTVTVEGCGTTSLQGDVKFAEVLEMMGAKVTWTETSVTVTGPPREPFGRKHLKAIDVNMNKMPDAAMTLAVVALFADGPTAIRDVASWRVKETERMVAICTELTKLGASVEEGPDYCIITPPPKLNVTAIDTYGDHRMAMAFSLAACAEVPVTIRDPGCTRKTFPDYFDVLSTFVKN

SEQ ID NO: 8 - protein, 445 aa, Zea mays
MRKWEEIVLQPIKEISGTVKLPGSKSLSNRILLLAGLAEGTTVVDNLLNSEDVHYMLGALRTLGLSVEHDKAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVKAERSDSWDRFYIKGGQSYLSPKNAYVEGDASSASYFLAGAAITGGTVTVEGCGTTSLQGDVKFAEVLEMMGAKVTWTEASVTVTGPPREPFGRKHLKAIDVNMNKMPDAAMTLAVVALFADGPTAIRDVGSWRVKETERMVAIRTELTKLGASVEEGPDYCIITPPEKLNVTAIDTYGDHRMAMAFSLAACAEVPVTIRDPGCTRKTFPDYFDVLSTFVKN

SEQ ID NO: 9 - protein, 445 aa, Zea mays
MRGWEEIVLQPIKEISGTVKLPGSKSLSNRILLLAALAEGTTVVDNLLNSEDVHYMLGALRTLGLSVEHDKAAKRAVVVGCGGRFPVEDAKEEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVKAEHNDSWDRFYIKGGQKYESPKNAYVEGDASSASYFLAGAAITGGTVTVEGCGTTSLQGDVKFAEVLEMMGAKVTWTETSVTVSGPPREPFGRKHLKAIDVNMNKMPDAAMTLAVVALFADGPTAIRDVASWRVKETERMVAICTELTKLGASVEEGPDYCIITPPPKLNVTAIDTYGDHRMAMAFSLAACAEVPVTIRDPGCTRKTFPDYFDVLSTFVKN

SEQ ID NO: 10 - protein, 445 aa, Zea mays
MRGWEEIVLQPIKEISGTVKLPGSKSLSNRILLLAALSEGTTVVDNLLNSEDVHYMLGALRTLGLSVEHDKAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVKAEHSDSWDRFYIKGGQKYESPKNAYVEGDASSASYFLAGAAITGGTVTVEGCGTTSLQGDVKFAEVLEMMGAKVTWTETSVTVTGPPREPFGRKHLKAIDVNMNKMPDAAMTLAVVALFADGPTAIRDVASWRVKETERMVAIRTELTKLGASVEEGPDYCIITPPPKLNVTAIDTYGDHRMAMAFSLAACAEVPVTIRDPGCTRKTFPDYFDVLSTFVKN

SEQ ID NO: 11 - protein, 445 aa, Zea mays
MAGAEEIVLQPIKEISGTVKLPGSKSLSNRILLLAALSEGTTVVDNLLNSEDVHYMLGALRTLGLSVEADKAAKRAVVVGCGGKFPVEDAKEEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKESERMVAIRTELTKLGASVEEGPDYCIITPPEKLNVTAIDTYGDHRMAMAFSLAACAEVPVTIRDPGCTRKTFPDYFDVLSTFVKN

SEQ ID NO: 12 - protein, 445 aa, Zea mays
MAGAEEIVLQPIKEISGTVKLPGSKSLSNRILLLAALSEGTTVVDNLLNSEDVGYMLGALRTLGLSVEADKAAKRAVVVGCGGKFPVEDAKEEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLVLMERFGVKAYHSDSWDRFYIKGGQKYKSPKNAYVEGDASSASYFLAGAAITGGTVTVEGCGTTSLQGDVKFAEVLEMMGAAVTWTETSVTVTGPPREPFGRKHLKAIDVNMNKMPDAAMTLAVVALFADGPTAIRDVASWRVKESERMVAIRTELTKLGASVEEGPDYCIITPPEKLNVTAIDTYGDHRMAMAFSLAACAEVPVTIRDPGCTAKTFPDYFDVLSTFVKN

SEQ ID NO: 13 - protein, 452 aa, Glycine max
MAAAEKPSTSPEIVLEPIKDFSGTITLPGSKSLSNRILLLAALSEGTTVVDNLLYSEDIHYMLGALRTLGLRVEDDKTTKQAIVEGCGGLFPTSKESKDEINLFCGNAATAMRPLTAAVVAAGGNASYVLDGVPRMRERPIGDLVAGLKQLGADVDCFLGTNCPPVRVNGKGGLPGGKVKLSGSVSSQYLTALLMAAPLALGDVEIEIVDKLISVPYVEMTLKLMERFGVSVEHSGNWDRFLVHGGQKYKSPGNAFVEGDASSASYLLAGAAITGGTITVNGCGTSSLQGDVKFAEVLEKMGAKVTWSENSVTVSGPPRDFSGRKVLRGIDVNMNKMPDVAMTLAVVALFANGPTAIRDVASWRVKESERMIAICTELRKLGATVEEGPDYCVITPPEKLNVTAIDTYGDHRMAMAFSLAACGDVPVTIKDPGCTRKTFPDYFEVLERLTKH

SEQ ID NO: 14 - protein, 452 aa, Glycine max
MAAAEKPSTSPEIVLEPIKDFSGTITLPGSKSLSNRILLLAALSEGTTVVDNLLYSEDIHYMLGALRTLGLRVEDDKTTKQAIVEGCGGLFPTSKESKDEINLFCGNAATAMRPLTAAVVAAGGNASYVLDGVPRMRERPIGDLVAGLKQLGADVDCFLGTNCPPVRVNGKGGLPGGKVKLSGSVSSQYLTALLMAAPLALGDVEIEIVDKLISVPYVEMTLKLMERFGVSVEHSGNWDRFLVHGGQKYKSPGNAFVEGDASSASYLLAGAAITGGTITVNGCGTSSLQGDVKFAEVLEKMGAKVTWSENSVTVSGPPRDFSGRKVLRGIDVNMNKMPDAAMTLAVVALFANGPTAIRDVASWRVKESERMIAICTELRKLGATVEEGPDYCVITPPEKLNVTAIDTYGDHRMAMAFSLAACGDVPVTIKDPGCTRKTFPDYFEVLERLTKH

SEQ ID NO: 15 - protein, 452 aa, Glycine max; identical to SEQ ID NO: 14

SEQ ID NO: 16 - protein, 446 aa, Oryza sativa
MRKWEEIVLQPIREISGAVQLPGSKSLSNRILLLSALSEGTTVVDNLLNSEDVHYMLEALKALGLSVEHDKVAKRAVVVGCGGRFPVEKDAKGEVQLFCGNAATAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISLPYVEMTLRLMERFGVKAEHSDSWDRFYIKGGQKYESPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPPKLNITAIDTYGDHRMAMAFSLAACADVPVTIRDPGCTRKTFPNYFDVLSTFVRN

SEQ ID NO: 17 - protein, 446 aa, Oryza sativa; identical to SEQ ID NO: 16

SEQ ID NO: 18 - protein, 446 aa, Oryza sativa
MAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFCGNAATAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKESERMVAIRTELTKLGASVEEGPDYCIITPPEKLNITAIDTYGDHRMAMAFSLAACADVPVTIRDPGCTRKTFPNYFDVLSTFVRN

SEQ ID NO: 19 - protein, 446 aa, Oryza sativa
MAKAEEIVLQPIREISGAVQLPGSKSLSNRILLLSALSEGTTVVDNLLNSEDVGYMLEALKALGLSVEADKVAKRAVVVGCGGKFPVEKDAKEEVQLFCGNAATAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLVLMERFGVKAYHSDSWDRFYIKGGQKYKSPGNAYVEGDASSASYFLAGAAITGGTVTVQGCGTTSLQGDVKFAEVLEMMGAAVTWTDTSVTVTGPPREPYGKKHLKAVDVNMNKMPDAAMTLAVVALFADGPTAIRDVASWRVKESERMVAIRTELTKLGASVEEGPDYCIITPPEKLNITAIDTYGDHRMAMAFSLAACADVPVTIRDPGCTAKTFPNYFDVLSTFVRN

SEQ ID NO: 20 - protein, 451 aa, Glycine max
AAAEKPSTSPEIVLEPIKDFSGTITLPGSKSLSNRILLLAALSEGTTVVDNLLYSEDIHYMLGALRTLGLRVEDDKTTKQAIVEGCGGLFPTSKESKDEINLFLGNAGTAMRPLTAAVVAAGGNASYVLDGVPRMRERPIGDLVAGLKQLGADVDCFLGTNCPPVRVNGKGGLPGGKVKLSGSVSSQYLTALLMAAPLALGDVEIEIVDKLISVPYVEMTLKLMERFGVSVEHSGNWDRFLVHGGQKYKSPGNAFVEGDASSASYLLAGAAITGGTITVNGCGTSSLQGDVKFAEVLEKMGAKVTWSENSVTVSGPPRDFSGRKVLRGIDVNMNKMPDVAMTLAVVALFANGPTAIRDVASWRVKETERMIAICTELRKLGATVEEGPDYCVITPPEKLNVTAIDTYDDHRMAMAFSLAACGDVPVTIKDPGCTRKTFPDYFEVLERLTKH

SEQ ID NO: 21 - protein, 526 aa, Glycine max
MAQVSRVHNLAQSTQIFGHSSNPNEPKSANSVSLRPRLWGPSKSRILVHKTGSLMGNFNAGKGNSGMFKVSASVAAAAEKPSTAPEIVLEPIKDISGTITLPGSKSLSNRILLLAALSEGTTVVDNLLYSEDIHYMLGALRTLGLRVEDDQTTKQAIVEGCGGLFPTIKESKDEINLFLGNAGTAMRPLTAAVVAAGGNASYVLDGVPRMRERPIGDLVAGLKQLGADVDCFLGTNCPPVRVNGKGGLPGGKVKLSGSISSQYLTALLMAAPLALGDVEIEIVDKLISVPYVEMTLKLMERFGVSVEHSGNWDKFLVHGGQKYKSPGNAFVEGDASSASYFLAGAAVTGGTITVNGCGTNSLQGDVKFAEVLEKMGAKVTWSENSVTVTGPPQDSSGQKVLQGIDVNMNKMPDVAMTLAVVALFANGQTAIRDVASWRVKETERMIAICTELRKLGATVEEGPDYCVITPPEKLNVTAIDTYDDHRMAMAFSLAACGDVPVTIKDPGCTRKTFPDYFEVLERFTRH

SEQ ID NO: 22 - protein, 446 aa, Oryza sativa
MAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGTAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 23 - protein, 445 aa, Sorghum halapense
AAGAEEIVLQPIKEISGTVKLPGSKSLSNRILLLAALSEGTTVVDNLLNSEDVHYMLGALNTLGLSVEADKVAKRAVVVGCGGKFPVEDAKEEVQLFLGNAGTAMRPLTAAVTAAGGNATYVLDGVPRMRERPIGDLVVGLKQLGADVDCFLGTDCPPVRINGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPRQPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 24 - protein, 509 aa, Helianthus sp.
MATHISNVAHNLQTPNFISKSPNPCSKLSPFLSFGSKYKTPSTQISVSSNNRKLFTVSASAATTAAVPEIVLQPIKEISGTVNLPGSKSLSNRILLLAALSEGTTVVDNLLNSDDVHYMLGALRTLGLRVEEDGAIKRAIVEGCGGVFPVGKEAKDEIQLFLGNAGTAMRPLTAAVTAAGGNSSYILDGVPRMRERPIGDLVTGLKQLGADVDCFLGTNCPPVRVVGGGGLPGGKVKLSGSISSQYLTALLMASPLALGDVEIEIIDKLISIPYVDMTIKLMERFGVSVEHSDSWDRFFIKGGQKYKSPGNAYVEGDASSASYFLAGAAITGGTITVEGCGTSSLQGDVKFAEVLGQMGAEVTWTENSVTVRGPPRDASGRKHLRAVDVNMNKMPDVAMTLAVVALYADGPTAIRDVASWRVKETERMIAICTELRKLGATVEEGPDYCVITPPERLNVTAIDTYDDHRMAMAFSLAACADVPVTIKDPACTRKTFPDYFEVLQRFTKH

SEQ ID NO: 25 - protein, 447 aa, Triticum aestivum
APSGAEEVVLQPIREISGAVQLPGSKSLSNRILLLSALSEGTTVVDNLLNSEDVHYMLEALEALGLSVEADKVAKRAVVVGCGGRFPVEKDAQEEVKLFLGNAGTAMRPLTAAVVAAGGNATYVLDGVPRMRERPIGDLVVGLQQLGADADCFLGTNCPPVRINGKGGLPGGKVKLSGSISSQYLSSLLMAAPLALEDVEIEIIDKLISVPYVEMTLKLMERFGVTAEHSDSWDRFYIKGGQKYKSPGNAYVEGDASSASYFLAGAAITGGTVTVEGCGTTSLQGDVKFAEVLEMMGAKVTWTDTSVTVTGPPRQPFGRKHLKAVDVNMNKMPDVAMTLAVVALFADGPTAIRDVASWRVKETERMVAIRTELTKLGATVEEGPDYCIITPPEKLNITAIDTYDDHRMAMAFSLAACAEVPVTIRDPGCTRKTFPNYFDVLSTFVKN

SEQ ID NO: 26 - protein, 446 aa, Brassica rapa
AEKASEIVLQPIREISGLIKLPGSKSLSNRILLLAALSEGTTVVDNLLNSDDINYMLDALKKLGLNVERDRVNNRAVVEGCGGIFPASLDSKSDIELYLGNAGTAMRPLTAAVTAAGGNASYVLDGVPRMRERPIGDLVVGLKQLGADVECTLGTNCPPVRVNANGGLPGGKVKLSGSISSQYLSALLMAAPLALGDVEIEIIDKLISVPYVEMTLKLMERFGVSAEHSDSWDRFFVKGGQKYKSPGNAYVEGDASSASYFLAGAAITGETVTVEGCGTTSLQGDVKFAEVLEKMGCKVSWTENSVTVTGPSRDAFGMRHLGAVDVNMDKMPDVAMTLAVVALFADGPTTLRDVASWRVKETERMIAICTELRKLGATVEEGSDYCVITPPAKVKPAEIDTYDDHRMAMAFSLAACADVPVTIKDPGCTRKTFPDYFQVLESITKH

SEQ ID NO: 27 - protein, 448 aa, Solanum lycopersicum
ATAEKPHEIVLEPIKDISGTVKLPGSKSLSNRILLLAALSEGRTVVDNLLSSDDIHYMLGALKTLGLHVEDDNENQRAIVEGCGGQFPVGKKSEEEIQLFLGNAGTAMRPLTAAVTVAGGHSRYVLDGVPRMRERPIGDLVDGLKQLGAEVDCSLGTNCPPVRIVSKGGLPGGKVKLSGSISSQYLTALLMAAPLALGDVEIEIIDKLISVPYVEMTLKLMERFGVFVEHSSGWDRFLVKGGQKYKSPGKAFVEGDASSASYFLAGAAVTGGTVTVEGCGTSSLQGDVKFAEVLEKMGAEVTWTENSVTVKGPPRNSSGMKHLRAIDVNMNKMPDVAMTLAVVALFADGPTTIRDVASWRVKETERMIAICTELRKLGATVVEGSDYCIITPPEKLNVTEIDTYDDHRMAMAFSLAACADVPVTIKDPGCTRKTFPDYFEVLQKYSKH

SEQ ID NO: 28 - protein, 448 aa, Solanum tuberosum
ATAEKPHEIVLEPIKDISGTVKLPGSKSLSNRILLLAALSEGRTVVDNLLSSDDIHYMLGALKTLGLHVEDDNENQRAIVEGCGGQFPVGKKSEEEIQLFLGNAGTAMRPLTAAVTVAGGHSRYVLDGVPRMRERPIGDLVDGLKQLGAEVDCSLGTNCPPVRIVSKGGLPGGKVKLSGSISSQYLTALLMAAPLALGDVEIEIIDKLISVPYVEMTLKLMERFGVFVEHSSSWDRFLVKGGQKYKSPGKAFVEGDASSASYFLAGAAVTGGTVTVEGCGTSSLQGDVKFAEVLEKMGAEVTWTENSVTVKGPPRNSSGMKHLRAIDVNMNKMPDVAMTLAVVALFADGPTTIRDVASWRVKETERMIAICTELRKLGATVVEGSDYCIITPPEKLNVTEIDTYDDHRMAMAFSLAACADVPVTIKDPGCTRKTFPDYFEVLQKYSKH

SEQ ID NO: 29 - DNA, 1362 bp, Zea mays
gctggtgcggaggaaattgtgctgcagcctatcaaagaaatctctggtaccgtgaagttgccgggctccaaaagcttgtctaaccgtatcctgctgctggccgcattgagcgagggtacgaccgttgtggacaatctgctgaatagcgaagatgtccactatatgctgggtgcactgcgtaccttgggcctgagcgttgaggcggacaaggccgcgaagcgcgctgttgttgtcggttgcggcggtaagttcccggttgaggatgccaaggaagaggttcaactgttcttgggcaatgctggtaccgcaatgcgtcctctgacggcggcagtgaccgcagctggcggtaatgctacctacgtcctggacggcgtgccgcgtatgcgcgagcgtccgattggcgatctggttgtgggtctgaagcagttgggcgcagatgtcgactgttttctgggcacggactgcccgcctgtccgcgtgaacggtattggtggcctgccgggtggtaaagtcaagctgagcggcagcattagcagccaatacctgtccgcattgctgatggcggcaccgctggccctgggcgacgtagaaattgagatcatcgacaagctgatttccatcccgtacgtcgaaatgacgctgcgtctgatggagcgtttcggcgtgaaggcggaacatagcgacagctgggaccgtttttacatcaaaggtggtcagaagtacaaaagcccgaaaaacgcgtacgttgagggcgatgcgagcagcgccagctatttcttggcgggtgcggcgatcacgggcggtacggtgaccgttgagggttgcggtacgacgtccctgcaaggcgacgtgaaattcgctgaggttctggaaatgatgggtgcgaaggtcacttggaccgagactagcgttaccgtgaccggtccaccgcgcgaaccgttcggccgcaagcatctgaaagcgattgatgtgaacatgaataagatgccggatgtcgcgatgaccctggccgtcgttgccctgtttgcggacggtccgaccgcgattcgtgatgttgcatcgtggcgcgtgaaagagactgaacgtatggtggcgattcgtaccgagctgaccaaactgggtgcaagcgttgaagagggtccggattattgtattatcacgccgccagaaaaactgaacgttacggccatcgacacctatgatgaccaccgcatggcgatggcgttcagcctggcggcatgcgcggaggtgccggtcaccattcgtgatccgggttgtacccgtaaaacctttccggactattttgatgttctgagcacctttgtcaaaaactacccttacgatgttcctgattacgcttaa

SEQ ID NO: 30 - DNA, 186 bp, Arabidopsis thaliana
atggcggcaatggcgacgaaggctgctgcgggaaccgtgtctttggaccttgccgctccaagcagaagacaccacagaccaagctctgctagacctccattcagacctgcagttagggggttacgtgctcctggaaggagagttatcgctgcccctccagcagctgcagcggcagccgcagttcag

SEQ ID NO: 31 - DNA, 162 bp, Artificial
  Chloroplast transit peptide
  transit_peptide (1)..(162)
atggctgcaactactcttacatctgctttacctggtgctttcagttcatcccagagaccaagtgctccttttaacttacaaagatcacctagagtgcttagaaggttcaacagaaagacaggacgtcaacctcgtggacttgttcgtgctgcaaaggctcaa

SEQ ID NO: 32 - DNA, 993 bp, Arabidopsis thaliana
agatcgataatctatttttgttttaaataggagaaaacaaaaattgattttttgtttggtttttgttttggtgtctaaatatatgtagattttctagtttctagtccttcgcttccaagttcctcgatggctcaatgccaatttagtcagataagattatgctgcaactgctagagcgactctctcctacttcattttagtccatactagttcattttatctccaagaaaatgactccgttttgcttcaaagctcaaagatgattctttttttaatgggccctcttgaaaaatgggtaaaatcttggtctttttacagatagcaaacatatacaaattatggagaaaacaggattatattgatgcagcttcgttgtagacagacttgtagtcgttttcttcattttcccattcctctgctaaattattaaatccaacaaaaacaaattttcttctgttgcttgataaaatctatgtggaatatctatatctacacactactgaagaccaagaagtaaacattagtttgcctgatcttcacctacctcaaacgagaagtagaagtttttatggtgacttttgtatttagagaaacaatgggattccagtttaagttggctctttttaactttgctaacatatgcctgaaagtggaagacaagaacttggtttaagaaacctcaagcgattgcgattttgggtcttaagcttgaaaaaagtgttgtatggaacaaacaaactaacatatcggaacaagcttggctttggttttaaagctgatagataatggtcgaaccataaccggtatggcccaagatgttcatgttttttaaaactcaccaaagctatatcactaacccacacattcttgcagaaggttttagaatcacaaagcataactcacctacccctaaaccaactccaatttctctcctcctctattaaatctttctcaatcatctttctttgagtcttttgccttggaatcctgatc

SEQ ID NO: 33 - DNA, 1277 bp, Arabidopsis thaliana
cggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtcggtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttaagaagttgctgttaaaaaaaaaaaggggattaacaacaacaacaaaaaaagataaagaaaataataacaattactttaattgtagactaaaaaaacatagattttatcatgaaaaaaagagaaaagaaataaaaacttggatcaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttctttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtaccatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtcttttt

SEQ ID NO: 34 - DNA, 917 bp, Arabidopsis thaliana
atcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacc

SEQ ID NO: 35 - DNA, 1157 bp, Phaseolus vulgaris
aagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtggacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttca

SEQ ID NO: 36 - protein, 518 aa, Helianthus annuus
MAIHINNISNFTSNLTNTHNPNPSSKSSPSSFLSFGSNFNNPMMNLASVSCKQNDQKRSLAVAASVATTQKTSTAPEEIVLKPIKEISGTVNLPGSKSLSNRILLLAALAEGTTVVDNLLNSDDVHYMLGALRALGLNVEENGEIKRATVEGCGGVFPVGKEAKDEIQLFLGNAGTAMRPLTAAVTAAGGNSSYILDGVPRMRERPIGDLVTGLKQLGADVDCFLGTNCPPVRVAANGGLPGGKVKLSGSISSQYLTALLMAAPLALGDVEIEIIDKLISVPYVEMTLKLMERFGVSVEHSDSWDKFYVRGGQKYKSPGNAYVEGDASSASYFLAGAAITGGTVTVEGCGTSSLQGDVKFAEVLGQMGAEVTWTENSVTVRGPPRNASGRGHLRPVDVNMNKMPDVAMTLAVVALYADGPTAIRDVASWRVKETERMIAICTELRKLGATVEEGPDYCVITPPEKLNVTAIDTYDDHRMAMAFSLAACADVPVTIKDPGCTRKTFPDYFEVLERFTKH

SEQ ID NO: 37 - protein, 446 aa, Oryza sativa
MRKWEEIVLQPIREISGAVQLPGSKSLSNRILLLSALSEGTTVVDNLLNSEDVGYMLEALKALGLSVEHDEVAKRAVVVGCGGRFPVEKDAKGEVQLFCGNAATAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISLPYVEMTLRLMERFGVRAEHSDSWDRFYIKGGQKYESPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVGSWRVKETERMVAIRTELTKLGASVEEGPDYCIITPPPKLNITAIDTYGDHRMAMAFSLAACGDVPVTIRDPGCTRKTFPNYFDVLSTFVRN

SEQ ID NO: 38 - protein, 445 aa, Sorghum bicolor
MRKWEEIVLQPIKEISGTVKLPGSKSLSNRILLLAALSEGTTVVDNLLNSEDVGYMLGALNTLGLSVEHDEVAKRAVVVGCGGRFPVEDAKGEVQLFCGNAATAMRPLTAAVTAAGGNATYVLDGVPRMRERPIGDLVVGLKQLGADVDCFLGTDCPPVRINGIGGLPGGKVKLSGSISSQYLSALLMAAPLALGDVEIEIIDKLISLPYVEMTLRLMERFGVRAEHSDSWDRFYIKGGQKYESPKNAYVEGDASSASYFLAGAAITGGTVTVEGCGTTSLQGDVKFAEVLEMMGAKVTWTETSVTVTGPPRQPFGRKHLKAIDVNMNKMPDAAMTLAVVALFANGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 39 - protein, 446 aa, Helianthus annuus
MRKWEEIVLKPIKEISGTVNLPGSKSLSNRILLLAALAEGTTVVDNLLNSDDVGYMLGALRALGLNVEHNEEIKRATVEGCGGRFPVGKEAKGEIQLFCGNAATAMRPLTAAVTAAGGNSSYILDGVPRMRERPIGDLVTGLKQLGADVDCFLGTNCPPVRVAANGGLPGGKVKLSGSISSQYLTALLMAAPLALGDVEIEIIDKLISLPYVEMTLKLMERFGVRVEHSDSWDKFYVRGGQKYESPGNAYVEGDASSASYFLAGAAITGGTVTVEGCGTSSLQGDVKFAEVLGQMGAEVTWTENSVTVRGPPRNASGRGHLRPVDVNMNKMPDAAMTLAVVALYADGPTAIRDVGSWRVKETERMIAICTELRKLGATVEEGPDYCVITPPPKLNVTAIDTYGDHRMAMAFSLAACGDVPVTIKDPGCTRKTFPDYFEVLERFTKH

SEQ ID NO: 40 - protein, 455 aa, Vitis vinifera
MASVATKEKPRKWPEIVLQPIKEISGTITLPGSKSLSNRILLLAALSEGTTVVDNLLNSEDVGYMLGALRTLGLHVEHDEENKRVIVQGCGGRFPAGNGSVGEVQLFCGNAATAMRPLTAAVTAAGGNASYVLDGVPRMRERPIGDLVTGLKQLGADVNCFLGTNCPPVRVNGNGGLPGGKVKLSGSISSQYLTALLMAAPLALGDVEIEIIDKLISLPYVEMTLKLMERFGVRVEHSNTWDRFLIRGGQKYESPGNAFVEGDASSASYFLAGAAVTGGTVTVEGCGTSSLQGDVKFAEVLEQMGAKVSWMENSVTVTGPPRDSSGRKHLRAIDVNMNKMPDAAMTLAVVALYADGPTAIRDVGSWRVKETERMIAICTELRKLGATVEEGPDYCVITPPPKLNVTSIDTYGDHRMAMAFSLAACGDVPVTIKDPGCTRKTFPDYFEVLQRFTKH

SEQ ID NO: 41 - protein, 446 aa, Gossypium hirsutum
MRKWSEIVLQPINEISGTVKLPGSKSLSNRILLLAALSEGTTVVENLLNSDDVGHMLVALGKLGLYVKHDEEKKQAIVEGCGGRFPVGKGEGGEIELFCGNAATAMRPLTAAITAAGGNSSYVLDGVPRMRERPIGDLVTGLKQLGADVDCILGTNCPPVRIEGKGGLPGGKVKLSGSISSQYLTALLMAAPLALGDVEIEIIDKLISLPYVEMTMKLMERFGVRVEHTDSWDRFFIRGGQKYESPGNAYVEGDASSASYFLAGAAVTGGTVTVEGCGTSSLQGDVKFAEVLEMMGAKVTWTENSVTVTGPPRNSSGRKHLRAIDVNMNKMPDAAMTLAVVALYADGPTAIRDVGSWRVKETERMIAICTELRKLGATVEEGPDYCVITPPPKLNVTAIDTYGDHRMAMAFSLAACGEVPVTIKDPGCTRKTFPDYFEVLDRVTKH

SEQ ID NO: 42 - protein, 445 aa, Manihot esculenta
MRKWPEIVLQPINEISGVVTLPGSKSLSNRILLLAALSEGTTVVDNLLNSDDVGYMLGALRTLGLHVEHNEQLKRAVVEGCGGRFPVGRKSKGNVELFCGNAATAMRPLTAAVTAAGGNSSYILDGVPRMRERPIGDLVAGLKQLGADIECSSTNCPPVHVNGKGGLPGGKVKLSGSISSQYLTALLMAAPLALGDVEIEIIDKLISLPYVEMTLKLMERYGVRVKHTGSWDRFFIRGGQKYESPGNSFVEGDASSASYFLAGAAITGGTVTVEGCGTSSLQGDVKFAEVLEKMGAKVTWTENSVTVTGPPRSSPSEKHLRAIDVNMNKMPDAAMTLAVVALFADGPTAIRDVGSWRVKETERMVAICTELRKLGATVEEGPDYCVITPPPKLNIAEIDTYGDHRMAMAFSLAACGDVPVTIKDPGCTRKTFPDYFEVLERFTKQ

SEQ ID NO: 43 - protein, 446 aa, Glycine max
MRKWPEIVLEPIKDFSGTITLPGSKSLSNRILLLAALSEGTTVVDNLLYSEDIGYMLGALRTLGLRVEHDETTKQAIVEGCGGRFPTSKESKGEINLFCGNAATAMRPLTAAVVAAGGNASYVLDGVPRMRERPIGDLVAGLKQLGADVDCFLGTNCPPVRVNGKGGLPGGKVKLSGSVSSQYLTALLMAAPLALGDVEIEIVDKLISLPYVEMTLKLMERFGVRVEHSGNWDRFLVHGGQKYESPGNAFVEGDASSASYLLAGAAITGGTITVNGCGTSSLQGDVKFAEVLEKMGAKVTWSENSVTVSGPPRDFSGRKVLRGIDVNMNKMPDAAMTLAVVALFANGPTAIRDVGSWRVKETERMIAICTELRKLGATVEEGPDYCVITPPPKLNVTAIDTYGDHRMAMAFSLAACGDVPVTIKDPGCTRKTFPDYFEVLERLTKH

SEQ ID NO: 44 - protein, 446 aa, Triticum aestivum
MRKWEEVVLQPIREISGAVQLPGSKSLSNRILLLSALSEGTTVVDNLLNSEDVGYMLEALEALGLSVEHDEVAKRAVVVGCGGRFPVEKDAQGEVKLFCGNAATAMRPLTAAVVAAGGNATYVLDGVPRMRERPIGDLVVGLQQLGADADCFLGTNCPPVRINGKGGLPGGKVKLSGSISSQYLSSLLMAAPLALEDVEIEIIDKLISLPYVEMTLKLMERFGVRAEHSDSWDRFYIKGGQKYESPGNAYVEGDASSASYFLAGAAITGGTVTVEGCGTTSLQGDVKFAEVLEMMGAKVTWTDTSVTVTGPPRQPFGRKHLKAVDVNMNKMPDAAMTLAVVALFADGPTAIRDVGSWRVKETERMVAIRTELTKLGATVEEGPDYCIITPPPKLNITAIDTYGDHRMAMAFSLAACGEVPVTIRDPGCTRKTFPNYFDVLSTFVKN

SEQ ID NO: 45 - protein, 445 aa, Zea mays
MSKWEEIVLQPIKEISGTVKLPGSKSLSNRILLLAALSEGTTVVDNLLNSEDVGYMLGALRTLGLSVEHDEAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRAEHSDSWDRFYIKGGQKYESPKNAYVEGDASSASYFLAGAAITGGTVTVEGCGTTSLQGDVKFAEVLEMMGAKVTWTETSVTVEGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 46 - protein, 445 aa, Zea mays
MSKWEEIVLQPIKEISGTVKLPGSKSLSNRILLLAALSEGTTVVDNLLNSEDVGYMLGALRTLGLSVEHDEAAKRAVVVGCGGRFPVEDAKGEVQLFCGNAATAMRPLTAAVTAAGGRATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRVEHSDSWDRFYIKGGQKYESPKNAYVEGDASSASYFLAGAAITGGTVTVEGCGTTSLQGDVKFAEVLEMMGAKVTWTATSVTVTGPRREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 47 - protein, 445 aa, Zea mays
MRKWEEIVLQPIKEISGTVKLPGSKSLSNRILLLAALSEGTTVVDNLLNSEDVGYMLGALRTLGLTVEHDEAAKRAVVVGCGGRFPVGDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRAEHSDSWDRFYIKGGQRYESPKNAYVEGDASSASYFLAGAAITGGTVTVEGCGTTSLQGDVKFAEVLELMGAKVTWTPTSVTVTGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 48 - protein, 445 aa, Zea mays
MSKWEEIVLQPIKEISGTVKLPGSKSLSNRILLLAALSEGTTVVDNLLNSEDVGYMLGALRTLGLTVEHDEAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRAEHSDSWDRFYIKGGQKYESPKNAYVEGDASSASYFLAGAAITGGTVTVEGCGTTSLQGDVKFAEVLELMGAKVTWTETSVTVTGPRREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 49 - protein, 445 aa, Zea mays
MSKWEEIVLQPIKEISGTVKLPGSKSLSNRILLLAALSEGTTVVDNLLNSEDVGYMLGALRTLGLSVEHDEAAKRAVVVGCGGRFPVGDAKGEVQLFCGNAATAMRPLTAAVTAAGGNATYVLDGVPRMRERPIGPLVVGLKQLGADVDCFLGTDCPPVRVNGIGGLPGGKVKLSGSISSQYLSALLMAAPLALGDVEIEIIDKLISLPYVEMTLRLMERFGVRAEHSDSWDRFYIKGGQKYESPKNAYVEGDASSASYFLAGAAITGGTVTVEGCGTTSLQGDVKFAEVLELMGAKVTWTETSVTVTGPRREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 50 - protein, 445 aa, Zea mays; identical to SEQ ID NO: 49

SEQ ID NO: 51 - protein, 445 aa, Zea mays
MSKWEEIVLQPIKEISGTVKLPGSKSLSNRILLLAALSEGTTVVDNLLNSEDVGYMLGALRTLGLTVEHDEAAKRAVVVGCGGRFPVEDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRVEHSDSWDRFYIKGGQRYESPKNAYVEGDASSASYFLAGAAITGGTVTVEGCGTTSLQGDVKFAEVLEMMGAKVTWTETSVTVEGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 52 - protein, 445 aa, Zea mays
MSKWEEIVLQPIKEISGTVKLPGSKSLSNRILLLAALSEGTTVVDNLLNSEDVGYMLGALRTLGLTVEHDEAAKRAVVVGCGGRFPVGDAKGEVQLFCGNAATAMRPLTAAVTAAGGRATYVLDGVPRMRERPIGDLVVGLKQLGADVDCFLGTDCPPVRVNGIGGLPGGKVKLSGSISSQYLSALLMAAPLALGDVEIEIIDKLISRPYVEMTLRLMERFGVRAEHSDSWDRFYIKGGQKYESPKNAYVEGDASSASYFLAGAAITGGTVTVEGCGTTSLQGDVKFAEVLELMGAKVTWTPTSVTVTGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 53 - protein, 445 aa, Zea mays
MFKWEEIVLQPIKEISGTVKLPGSKSLSNRILLLAALSEGTTVVDNLLNSEDVGYMLGALRTLGLTVEHDEAAKRAVVVGCGGRFPVGDAKGEVQLFCGNAATAMRPLTAAVTAAGGNATYVLDGVPRMRERPIGDLVVGLKQLGADVDCFLGTDCPPVRVRGIGGLPGGKVKLSGSISSQYLSALLMAAPLALGDVEIEIIDKLISLPYVEMTLRLMERFGVRAEHSDSWDRFYIKGGQRYESPKNAYVEGDASSASYFLAGAAITGGTVTVEGCGTESLQGDVKFAEVLELMGAKVTWTPTSVTVTGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 54 - protein, 445 aa, Zea mays
MSKWEEIVLQPIKEISGTVKLPGSKSLSNRILLLAALSEGTTVVDNLLNSEDVGYMLGALRTLGLSVEHDEAAKRAVVVGCGGRFPVGDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRVEHSDSWDRFYIKGGQKYESPKNAYVEGDASSASYFLAGAAITGGTVTVEGCGTTSLQGDVKFAEVLEMMGAKVTWTETSVTVTGPWREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 55 - protein, 445 aa, Zea mays
MSKWEEIVLQPIKEISGTVKLPGSKSLSNRILLLAALSEGTTVVDNLLNSEDVGYMLGALRTLGLSVEHDEAAKRAVVVGCGGRFPVGDAKGEVQLFCGNAATAMRPLTAAVTAAGGRATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRVEHSDSWDRFYIKGGQRYESPKNAYVEGDASSASYFLAGAAITGGTVTVEGCGTTSLQGDVKFAEVLEMMGAKVTWTETSVTVEGPWREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 56 - protein, 445 aa, Zea mays
MSKWEEIVLQPIKEISGTVKLPGSKSLSNRILLLAALSEGTTVVDNLLNSEDVGYMLGALRTLGLSVEHDEAAKRAVVVGCGGRFPVEDAKGEVQLFCGNAATAMRPLTAAVTAAGGRATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRVEHSDSWDRFYIKGGQRYESPKNAYVEGDASSASYFLAGAAITGGTVTVEGCGTTSLQGDVKFAEVLELMGAKVTWTSTSVTVTGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 57 - protein, 445 aa, Zea mays
MRKWEEIVLQPIKEISGTVKLPGSKSLSNRILLLAALSEGTTVVDNLLNSEDVGYMLGALRTLGLSVEHDEAAKRAVVVGCGGRFPVGDAKGEVQLFCGNAATAMRPLTAAVTAAGGRATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRVEHSDSWDRFYIKGGQKYESPKNAYVEGDASSASYFLAGAAITGGTVTVEGCGTTSLQGDVKFAEVLEMMGAKVTWTETSVTVTGPVREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 58 - protein, 445 aa, Zea mays
MSKWEEIVLQPIKEISGTVKLPGSKSLSNRILLLAALSEGTTVVDNLLNSEDVGYMLGALRTLGLTVEHDEAAKRAVVVGCGGRFPVGDAKGEVQLFCGNAATAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISLPYVEMTLRLMERFGVRVEHSDSWDRFYIKGGQKYESPKNAYVEGDASSASYFLAGAAITGGTVTVEGCGTTSLQGDVKFAEVLELMGAKVTWTETSVTVAGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

SEQ ID NO: 59 - protein, 445 aa, Zea mays
MSKWEEIVLQPIKEISGTVKLPGSKSLSNRILLLAALSEGTTVVDNLLNSEDVGYMLGALRTLGLSVEHDEAAKRAVVVGCGGRFPVEDAKGEVQLFCGNAATAMRPLTAAVTAAGGNATYVLDGVPRMRERPIGDLVVGLKQLGADVDCFLGTDCPPVRVRGIGGLPGGKVKLSGSISSQYLSALLMAAPLALGDVEIEIIDKLISLPYVEMTLRLMERFGVRAEHSDSWDRFYIKGGQKYESPKNAYVEGDASSASYFLAGAAITGGTVTVEGCGTTSLQGDVKFAEVLELMGAKVTWTPTSVTVTGPPREPFGRKHLKAIDVNMNKMPDAAMTLAVVALFADGPTAIRDVGSWRVKETERMVAIRTELTKLGASVEEGPDYCIITPPPKLNVTAIDTYGDHRMAMAFSLAACGEVPVTIRDPGCTRKTFPDYFDVLSTFVKN

(19) United States
(12) Patent Application Publication
(10) Pub. No.: US 2020/0392528 A1
(43) Pub. Date: Dec. 17, 2020

(54) IMPROVED PLANT EPSP SYNTHASES AND METHODS OF USE

(71) Applicant: PIONEER HI-BRED INTERNATIONAL, INC., JOHNSTON, IA (US)

(72) Inventors: YUXIA DONG, SAN RAMON, CA (US); JIAN LU, ALAMEDA, CA (US); EMILY NG, SAN LEANDRO, CA (US); DANIEL SIEHL, MENLO PARK, CA (US); YUMIN TAO, FREMONT, CA (US)

(73) Assignee: PIONEER HI-BRED INTERNATIONAL, INC., JOHNSTON, IA (US)

(21) Appl. No.: 16/498,569

(22) PCT Filed: Mar. 21, 2018
(86) PCT No.: PCT/US18/23480

§ 371 Date: Sep. 27, 2019

Related U.S. Application Data
(60) Provisional application No. 62/478,636, filed on Mar. 30, 2017.

(51) Int. Cl. C12N 15/82; C12N 9/10

(52) U.S. Cl. CPC C12N 15/8275; C12Y 205/01019; C12N 9/1092

(57) ABSTRACT

Compositions and methods comprising polynucleotides and polypeptides having EPSP (5-enolpyruvylshikimate-3-phosphate) synthase (EPSPS) activity are provided. In specific embodiments, the sequence has an improved property, such as, but not limited to, improved catalytic capacity in the presence of the inhibitor, glyphosate. Further provided are nucleic acid constructs, plants, plant cells, explants, seeds and grain having the EPSPS sequences. Various methods of employing the EPSPS sequences are provided. Such methods include methods for producing a glyphosate tolerant plant, plant cell, explant or seed and methods of controlling weeds in a field containing a crop employing the plants and/or seeds disclosed herein.

### FIELD

The field relates to the field of molecular biology. More specifically, it pertains to sequences that confer tolerance to glyphosate.

### REFERENCE TO SEQUENCE LISTING SUBMITTED ELECTRONICALLY

The official copy of the sequence listing is submitted electronically via EFS-Web as an ASCII formatted sequence listing with a file named 7410PCT_ST25.txt created on Mar. 18, 2018 and having a size 207 kilobytes and is filed concurrently with the specification. The sequence listing contained in this ASCII formatted document is part of the specification and is herein incorporated by reference in its entirety.

### BACKGROUND

EPSP (5-enolpyruvylshikimate-3-phosphate) synthase is an enzyme that catalyzes the conversion of phosphoenolpyruvate and 3-phosphoshikimate to phosphate and 5-enolpyruvylshikimate-3-phosphate (EPSP), and it participates in the biosynthesis of the aromatic amino acids phenylalanine, tyrosine, and tryptophan. Glyphosate, the top selling herbicide in the world, acts a competitive inhibitor for phosphoenolpyruvate.

Glyphosate tolerant crops have been created by introducing glyphosate-insensitive 5-enolpyruvylshikimate-3-phosphate synthase (EPSPS) enzymes into plants. In one example, maize event NK603 uses EPSPS from Agrobacterium sp. strain CP4. The enzyme is highly insensitive to inhibition by glyphosate while retaining catalytic efficiency similar to native plant enzymes (Sikorski and Gruys. 1997. Acc. Chem. Res. 30:2-8). In another example, maize event GA21 uses a double mutant maize EPSPS in which threonine at position 103 is changed to isoleucine and proline at position 107 is changed to serine.

Plant EPSP synthases having kinetic properties that provide adequate tolerance to glyphosate and catalytic capacity to sustain normal rates of metabolic flux are desired.

### SUMMARY

Plant EPSP synthases (herein referred to as EPSPS) and the polynucleotides that encode them are provided herein. Methods for generating glyphosate tolerant plants that are tolerant to the plant EPSPS enzymes are also provided.

Polynucleotides are provided herein that encode plant EPSPS polypeptides that comprise G102A and at least one or more amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the plant EPSPS polypeptide comprises a sequence that is at least 90% identical to SEQ ID NO:2.

In certain embodiments, the polynucleotide encodes a plant EPSPS polypeptide that comprises a plant EPSPS polypeptide variant designated Zm D2c-A5 that comprises A2S, G3K, A4W, H54G, A69H, K71E, K84R, L98C, N162R, I208L, K224R, K243E, M293L, E302P, V333A, A354G, E391P, D402G, and A416G; or A2R, G3K, A4W, H54G, A69H, K71E, K84R, L98C, I208L, K224R, K243E, V333A, A354G, E391P, D402G, and A416G; or the polynucleotide encodes a plant EPSPS polypeptide that comprises H54G, L98C, R216V, E226Y, K297A, V333A, T361S, D402G, and R429A; or the polynucleotide encodes a plant EPSPS polypeptide that comprises L98C, T361S, and D402G; or the polynucleotide encodes a plant EPSPS polypeptide that comprises A2R, A4W, A69H, K84R, L98C, I208L, K243E, V333A, E391P, and D402G; or the polynucleotide encodes a plant EPSPS polypeptide that comprises A2R, G3K, A4W, A69H, K84R, L98C, I208L, K243E, V333A, A354G, E391P, and D402G; or the polynucleotide encodes a plant EPSPS polypeptide that comprises A2R, G3K, A4W, H54L, A69H, K84R, L98C, I208L, K243E, V333A, R368C, E391P, and D402G; the polynucleotide encodes a plant EPSPS polypeptide that comprises A2R, G3K, A4W, S38A, H54L, A69H, K84R, E92G, L98C, I208L, K243L, V333A, R368C, E391P, and D402G. In still other embodiments, the polynucleotide encodes the plant EPSPS polypeptide set forth in one of SEQ ID NOS: 3-12.

Also provided are recombinant DNA constructs comprising the polynucleotides disclosed herein; plant cells comprising in their genomes a polynucleotide disclosed herein or a recombinant DNA construct comprising such; and plants comprising in their genomes a polynucleotide disclosed herein or a recombinant DNA construct comprising such. In some embodiments, the plant cell is a maize cell. In some embodiments, the plant is maize.

Methods of generating glyphosate tolerant plants are provided herein. The methods comprise expressing in a regenerable plant cell a recombinant DNA construct comprising a polynucleotide operably linked to at least one regulatory sequence, wherein the polynucleotide encodes a plant EPSPS polypeptide that comprises G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the plant EPSPS polypeptide comprises a sequence that is at least 90% identical to SEQ ID NO:2; and generating a glyphosate tolerant plant that comprises in its genome the recombinant DNA construct. In some embodiments, the methods include expressing in a plant cell a recombinant DNA construct comprising a polynucleotide encoding a plant EPSPS polypeptide comprising G102A and at least two, at least three, or at least four amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid position set forth in SEQ ID NO:1 and wherein the plant EPSPS polypeptide comprises a sequence that is at least 90% identical to SEQ ID NO:2.

In other embodiments, the method comprises expressing in a plant cell a recombinant DNA comprising a polynucleotide that encodes a plant EPSPS polypeptide that comprises A2R, G3K, A4W, H54G, A69H, K71E, K84R, L98C, I208L, K224R, K243E, V333A, A354G, E391P, D402G, and A416G; or the polynucleotide encodes a plant EPSPS polypeptide that comprises H54G, L98C, R216V, E226Y, K297A, V333A, T361S, D402G, and R429A; or the polynucleotide encodes a plant EPSPS polypeptide that comprises L98C, T361S, and D402G; or the polynucleotide encodes a plant EPSPS polypeptide that comprises A2R, A4W, A69H, K84R, L98C, I208L, K243E, V333A, E391P, and D402G; or the polynucleotide encodes a plant EPSPS polypeptide that comprises A2R, G3K, A4W, A69H, K84R, L98C, I208L, K243E, V333A, A354G, E391P, and D402G; or the polynucleotide encodes a plant EPSPS polypeptide that comprises A2R, G3K, A4W, H54L, A69H, K84R, L98C, I208L, K243E, V333A, R368C, E391P, and D402G; the polynucleotide encodes a plant EPSPS polypeptide that comprises A2R, G3K, A4W, S38A, H54L, A69H, K84R, E92G, L98C, I208L, K243L, V333A, R368C, E391P, and D402G. In still other embodiments, the method comprises expressing in a plant cell a recombinant DNA comprising a polynucleotide that encodes the plant EPSPS polypeptide set forth in One of SEQ ID NOS: 3-12.

Methods of generating glyphosate tolerant plants are provided herein, in which an endogenous plant EPSPS gene (in a plant cell) is modified to encode a glyphosate tolerant EPSPS protein that comprises G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the endogenous plant EPSPS gene encodes a polypeptide comprising a sequence that is at least 90% identical to SEQ ID NO:2; and a glyphosate tolerant plant is grown from the plant cell. In some embodiments the modified endogenous plant EPSPS gene encodes a glyphosate tolerant EPSPS protein that comprises G102A and at least two, at least three, or at least four of the amino acid mutations.

In other embodiments, the modified endogenous plant EPSPS gene encodes a glyphosate tolerant EPSPS protein that comprises: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G; or a plant EPSPS polypeptide that comprises L98C, T361S, and D402G; or a plant EPSPS polypeptide that comprises A2R, A4W, A69H, K84R, L98C, I208L, K243E, V333A, E391P, and D402G; or a plant EPSPS polypeptide that comprises A2R, G3K, A4W, A69H, K84R, L98C, I208L, K243E, V333A, A354G, E391P, and D402G; or a plant EPSPS polypeptide that comprises A2R, G3K, A4W, H54L, A69H, K84R, L98C, I208L, K243E, V333A, R368C, E391P, and D402G; or a plant EPSPS polypeptide that comprises A2R, G3K, A4W, S38A, H54L, A69H, K84R, E92G, L98C, I208L, K243L, V333A, R368C, E391P, and D402G. In still other embodiments, the modified endogenous plant EPSPS gene encodes a glyphosate tolerant EPSPS protein that comprises the plant EPSPS polypeptide set forth in one of SEQ ID NOS: 3-12 and 45-59 or a modified EPSPS sequence as specified in the sequence listing and accompanying table.

The endogenous plant EPSPS gene may be modified by a CRISPR/Cas guide RNA-mediated system, a Zn-finger nuclease-mediated system, a meganuclease-mediated system, or an oligonucleobase-mediated system.

Polynucleotides that provide a guide RNA in a plant cell are provided herein in which the guide RNA targets an endogenous EPSPS gene of the plant cell and further comprises one or more polynucleotide modification templates to generate a modified endogenous EPSPS gene that encodes a plant EPSPS polypeptide comprising G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the endogenous plant EPSPS gene encodes a polypeptide comprising a sequence that is at least 90% identical to SEQ ID NO:2. In some embodiments, the polynucleotide construct comprises one or more polynucleotide modification templates to generate a modified endogenous EPSPS gene encoding a plant EPSPS polypeptide that comprises G102A and at least two, at least three, or at least four amino acid mutations selected from the group above, wherein each amino acid position corresponds to the amino acid mutation position set forth in SEQ ID NO: 1 and wherein the endogenous plant EPSPS gene encodes a polypeptide comprising a sequence that is at least 90% identical to SEQ ID NO: 2. In still other embodiments, one or more polynucleotide modification templates include sequences to generate a modified endogenous EPSPS gene encoding a plant EPSPS polypeptide that has the amino acid sequence set forth in one of SEQ ID NOS: 3-12 and 45-59.

Methods for producing glyphosate tolerant plants are provided herein in which a guide RNA, one or more polynucleotide modification templates, and one or more Cas endonucleases are provided to a plant cell. The Cas endonuclease(s) introduces a double strand break at an endogenous EPSPS gene in the plant cell, and the polynucleotide modification template(s) is used to generate a modified EPSPS gene that encodes a plant EPSPS polypeptide that comprises G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the endogenous plant EPSPS gene encodes a polypeptide comprising a sequence that is at least 90% identical to SEQ ID NO:2. A plant is obtained from the plant cell, and a glyphosate tolerant progeny plant is generated. In some embodiments, the one or more polynucleotide modification templates are used to generate a modified endogenous EPSPS gene encoding a plant EPSPS polypeptide that comprises G102A and at least two, at least three, or at least four amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid mutation position set forth in SEQ ID NO:1 and wherein the endogenous plant EPSPS gene encodes a polypeptide comprising a sequence that is at least 90% identical to SEQ ID NO:2.

Also provided herein are glyphosate tolerant maize plants that express an endogenous EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid position set forth in SEQ ID NO:1 and wherein the endogenous plant EPSPS gene encodes a polypeptide comprising a sequence that is at least 90% identical to SEQ ID NO:2. A glyphosate tolerant maize plant may express a plant EPSPS polypeptide having the sequence set forth in One of SEQ ID NOS: 3-12.

Also provided herein are glyphosate tolerant sunflower plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% identical to SEQ ID NO: 24 or 36. In an embodiment, a glyphosate tolerant sunflower plant expressed a polynucleotide that encodes an EPSPS polypeptide having an amino acid sequence that exhibits at least 90%, or 95% or 96% or 98% or 99% identity to SEQ ID NO: 39.

Also provided herein are glyphosate tolerant rice plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% or 95% or 96% or 98% or 99% identical to SEQ ID NO: 22. A glyphosate tolerant rice plant may express a plant EPSPS polypeptide having the sequence set forth in one of SEQ ID NOS:16-19 and 37.

Also provided herein are glyphosate tolerant sorghum plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% identical to SEQ ID NO: 23.

Also provided herein are glyphosate tolerant soybean plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% identical to SEQ ID NO:20 or 21. A glyphosate tolerant soybean plant may express a plant EPSPS polypeptide having the sequence set forth in one of SEQ ID NOS:13-15 and 43.

Also provided herein are glyphosate tolerant wheat plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% identical to SEQ ID NO:25.

Also provided herein are glyphosate tolerant Brassica rapa plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% identical to SEQ ID NO: 26.

Also provided herein are glyphosate tolerant tomato plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% identical to SEQ ID NO: 27.

Also provided herein are glyphosate tolerant potato plants that express an EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the analogous amino acid position set forth in SEQ ID NO:1 and wherein the plant EPSPS gene encodes a polypeptide comprising a sequence that is at least 90% identical to SEQ ID NO: 28.

Methods of weed control in which an effective amount of glyphosate is applied over a population of glyphosate tolerant plants provided herein are also provided. The plants may be maize, sunflower, rice, wheat, tomato, potato, oil seed rape, sorghum, or soy. The effective amount of glyphosate applied may be about 50 gram acid equivalent/acre to about 2000 gram acid equivalent/acre.

Polynucleotide modification templates comprising a partial EPSP synthase (EPSPS) sequence, wherein a polynucleotide modification template comprises one or more nucleotide mutations that correspond to G102A and to at least one or more amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO: 1, are also provided. Plant cells comprising a polynucleotide modification template presented herein, a guide RNA, and CRISPR/Cas9 endonuclease are also provided wherein said combination targets an endogenous maize EPSPS sequence that encodes an EPSPS polypeptide that is at least 90% identical to SEQ ID NO:2.

Also provided is a method of rapidly assaying catalytic efficiency of a plurality of enzyme variants in the presence of an inhibitor. The method includes (a) providing a plurality of enzyme variants; (b) providing the inhibitor; (c) providing the substrate; (d) performing a reaction involving the plurality of enzyme variants and the substrate, at no more than two different inhibitor concentrations; (e) measuring reaction rate at no more than two different inhibitor concentrations; and (f) calculating (kcat/KM)*KI of the plurality of enzyme variants. In some embodiments, one of the inhibitor concentrations is zero. In other embodiments, the substrate is at a concentration that is substantially similar to Michaelis-Menten constant (KM) of a parental enzyme for the enzyme variant. In still other embodiments, the enzyme is at a sufficient concentration to result in a substantially linear reaction rate at the two different inhibitor concentrations. In still other embodiments, one of the inhibitor concentrations is sufficient to result in at least about 50% inhibition. In still other embodiments, the assay is performed in a high-throughput system. In still other embodiments, the catalytic capacity in the presence of the inhibitor is estimated by obtaining a numerical value for (kcat/KM)*KI, wherein kcat is maximum enzyme turnover rate, KM is Michaelis-Menten constant and KI is inhibitor dissociation constant. In some embodiments, the substrate is PEP; the inhibitor is glyphosate; and the plurality of enzyme variants are EPSPS enzyme variants. In some embodiments, the enzyme and the substrate concentrations are the same, at the two inhibitor concentrations.

### BRIEF DESCRIPTION OF THE DRAWINGS

FIG. 1A and FIG. 1B show a multiple sequence alignment alignment of maize and rice EPSPS amino acid sequences. Identical residues are shown with grey background. Note that the maize native amino acid sequence has an added Met (M) at the N-terminus which is not generally known to be present in the endogenous maize EPSPS processed mature protein without a chloroplast transit peptide (CTP). Th SEQ ID NOs represented in FIG. 1A and FIG. 1B are Zm Native (SEQ ID NO: 1), Zm F3 (SEQ ID NO: 11), Os AF4 (SEQ ID NO: 22), Os F3 (SEQ ID NO: 18), Os F3-88 (SEQ ID NO: 19), Os D2 (SEQ ID NO: 16), Os D2-67 (SEQ ID NO: 17), Zm D2 (SEQ ID NO: 10), Zm D2-67 (SEQ ID NO: 5) and Zm F3-88 (SEQ ID NO: 12).

FIG. 2 shows sequence of optimization pathway to generate improved EPSPS variants. Boxes indicate an EPSPS variant with number of mutations in parentheses. Arrows indicate an optimization process (saturation mutagenesis or combinatorial library). Key desensitizing mutation(s) are also shown. The table specifies the screening procedure for the adjacent library. ¹The vector used for expression in E. coli, described in Methods; “low copy” indicates that the ori is exchanged with that of pSC101, generating ˜5 copies rather than ˜20. ²Amendment added to the minimal basal medium described herein. ³Combi: Combinatorial library of the diversity indicated. ⁴Diversity: The neutral or beneficial substitutions identified by saturation mutagenesis. ⁵pmbn: Polymyxin B-sulfate nonapeptide, supplied at 1 mg/L. ⁶Backbone: The amino acid sequence upon which the combinatorial library is built. ⁷H6-C2-native backcross. ⁸betaine: Supplied at 1 mM. ⁹k_{gly} is enzyme turnover, min⁻¹, under simulated in vivo application conditions (30 μM PEP, 30 μM S3P and 1 mM glyphosate).

FIG. 3 shows the amino acid substitutions present in variants in the progressive optimization of maize EPSPS for activity in the presence of glyphosate. Amino acids that differ from that in the native enzyme are shaded. The positions shown in the the table correspond to the native maize EPSPS variant without the Met added at position 1 compared to other tables/sequences.

FIG. 4 shows the progressive fitness resulting from optimization of maize EPSPS with the G101A mutation. Note that the numbering refers to the relative position of native maize EPSPS without the N-terminal Met.

FIG. 5 demonstrates the improvements in k_{cat} and selectivity (Ki/Km) on fitness of optimized maize EPSPS with the G101A mutation. Note that the numbering refers to the relative position of native maize EPSPS without the N-terminal Met.

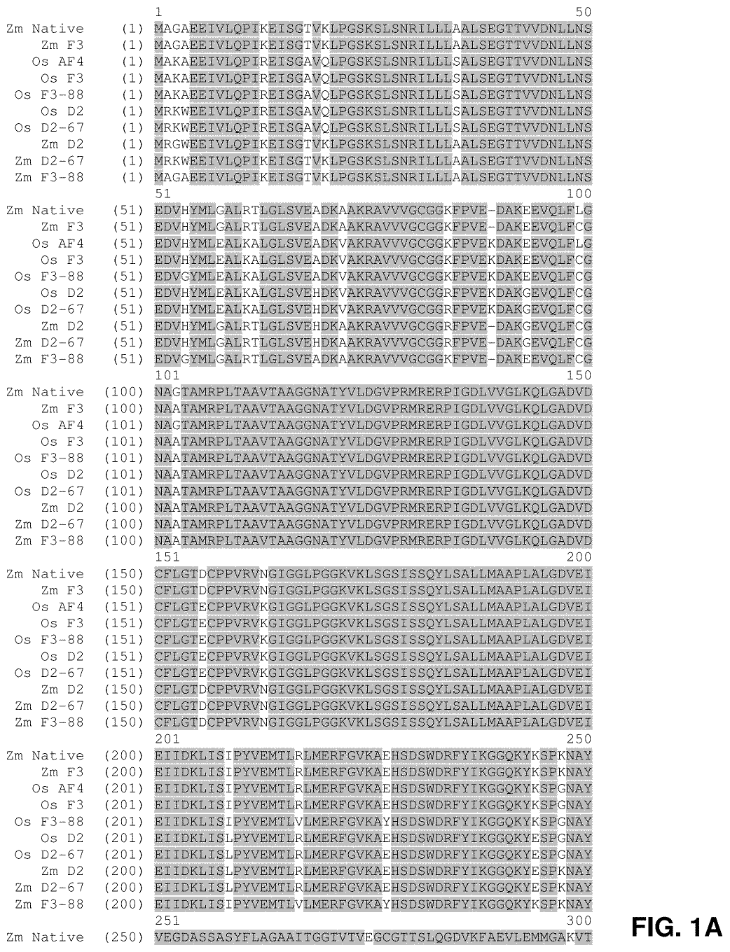

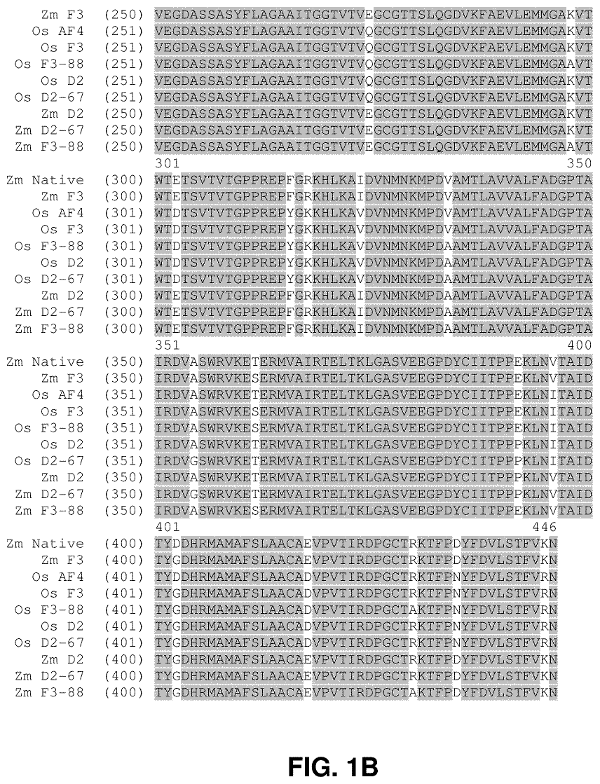

### BRIEF DESCRIPTION OF THE SEQUENCE LISTING

The sequence descriptions and Sequence Listing attached hereto comply with the rules governing nucleotide and/or amino acid sequence disclosures in patent applications as set forth in 37 C.F.R. § 1.821 1.825. The Sequence Listing contains the one letter code for nucleotide sequence characters and the three letter codes for amino acids as defined in conformity with the IUPAC IUBMB standards described in Nucleic Acids Res. 13:3021 3030 (1985) and in the Biochemical J. 219 (2):345 373 (1984) which are herein incorporated by reference. The symbols and format used for nucleotide and amino acid sequence data comply with the rules set forth in 37 C.F.R. § 1.822.

| SEQ | | |
|---|---|---|
| ID | | |
| NO | Crop | Sequence Description |
| 1 | Maize | Amino acid sequence of an expressed protein |
| | | obtained by cloning a synthetic EPSP synthase |
| | | (in which the nucleotide sequence of the gene |
| | | encoding SEQ ID NO: 2 was modified to add an |
| | | N-terminal methionine to SEQ ID NO: 2 for |
| | | expression in E. coli) into an expression |
| | | vector. SEQ ID NO: 1 is to be used herein |
| | | as a reference EPSPS sequence |
| 2 | Maize | Amino acid sequence of a maize EPSPS polypeptide |
| | | (444 amino acid protein) presented as GenBank |
| | | entry CAA44974.1 (NCBI GI No. 1524383); used to |
| | | refer to an endogenous maize EPS PS sequence |
| 3 | Maize | Zm D2-3P124 |
| 4 | Maize | Zm D2-68 |
| 5 | Maize | Zm D2-67 |
| 6 | Maize | Zm D2-82 |
| 7 | Maize | Zm D2-64 |
| 8 | Maize | Zm D2-28 |
| 9 | Maize | Zm D2-15 |
| 10 | Maize | Zm D2 |
| 11 | Maize | Zm F3 |
| 12 | Maize | Zm F3-88 |
| 13 | Soy | Gm F3 (includes N-terminal methionine; mature |
| | | form) |
| 14 | Soy | Gm F3-V340A (includes N-terminal methionine; |
| | | mature form) |
| 15 | Soy | Gm F3-02-A7 (includes N-terminal methionine; |
| | | mature form) |
| 16 | Rice | Os D2 |
| 17 | Rice | Os D2-67 |
| 18 | Rice | Os F3 |
| 19 | Rice | Os F3-88 |
| 20 | Soy | Gm EPSPS XP_00351 native EPSPS; mature form; |
| | | does not include the N-terminal Met |
| 21 | Soy | Gm XP_00352, NC_016090.2 native EPSPS |
| 22 | Rice | Os Native AF413082 |
| 23 | Sorghum | Sorghum halapense H6T5X2 native EPSPS |
| 24 | Sunflower | Helianthus annuus native EPSPS 1, |
| | | full length with CTP, 509aa |
| 25 | Wheat | Wheat ACH72672.1 native EPSPS |
| 26 | Brassica | Brassica rapa M4FGU1 native EPSPS |
| 27 | Tomato | Sol lyco K4AZ59 native EPSPS |
| 28 | Potato | Sol tuberosum M1CGC9 native EPSPS |
| 29 | Maize | maize EPSPS (CAA44974) DNA |
| 30 | Arabidopsis | DNA sequence coding for the chloroplast transit |
| | | peptide from Arabidopsis EPSPS |
| 31 | Artificial | Artificial CTP termed 6H1 |
| 32 | Arabidopsis | native Arabidopsis EPSPS promoter |
| | | (AT1G48860) |
| 33 | Arabidopsis | Ubiquitin-3 promoter; DNA |
| 34 | Arabidopsis | Ubiquitin-10 promoter |
| 35 | Phaseolus | Phaseolin terminator |
| | vulgaris | |
| 36 | Sunflower | Helianthus annuus EPSPS 2, full |
| | | length with CTP, 518 aa |
| 37 | Oryza | Rice EPSPS sequence with maize D2-124 mutations |
| | sativa; | mapped |
| | D2-124 | |
| 38 | Sorghum | Sorghum EPSPS sequence with maize |
| | bicolor, | D2-124 mutations mapped |
| | D2-124 | |
| 39 | Helianthus | Sunflower EPSPS sequence with maize D2-124 |
| | annuus; | mutations mapped |
| | D2-124 | |
| 40 | Vitis | Grapevine EPSPS sequence with maize D2-124 |
| | vinifera; | mutations mapped |
| | D2-124 | |
| 41 | Gossypium | Cotton EPSPS sequence with maize D2-124 |
| | hirsutum; | mutations mapped |
| | D2-124 | |
| 42 | Manihot | Cassava EPSPS sequence with maize D2-124 |
| | esculenta; | mutations mapped |
| | D2-124 | |
| 43 | Glycine | Soybean EPSPS sequence with maize D2-124 |
| | max; D2-124 | mutations mapped |
| 44 | Triticum | Wheat EPSPS sequence with maize D2-124 |
| | aestivum; | mutations mapped |
| | D2-124 | |
| 45 | Maize | Zm D2C-200 maize EPSPS variant |
| 46 | Maize | Zm D2C-152 maize EPSPS variant |
| 47 | Maize | Zm D2C-164a maize EPSPS variant |
| 48 | Maize | Zm D2C-171 maize EPSPS variant |
| 49 | Maize | Zm D2C-178 maize EPSPS variant |
| 50 | Maize | Zm D2C-230 maize EPSPS variant |
| 51 | Maize | Zm D2C-238 maize EPSPS variant |
| 52 | Maize | Zm D2C-106 maize EPSPS variant |
| 53 | Maize | Zm D2C-116 maize EPSPS variant |
| 54 | Maize | Zm D2C-118 maize EPSPS variant |
| 55 | Maize | Zm D2C-158 maize EPSPS variant |
| 56 | Maize | Zm D2C-170 maize EPSPS variant |
| 57 | Maize | Zm D2C-171 maize EPSPS variant |
| 58 | Maize | Zm D2C-173 maize EPSPS variant |
| 59 | Maize | Zm D2c-A5 maize EPSPS variant |

### DETAILED DESCRIPTION

### I. Compositions

### A. EPSP Synthase Polynucleotides and Polypeptides

Various methods and compositions are provided which employ polynucleotides and polypeptides having EPSP synthase (EPSPS) activity. Such EPSPS polypeptides include those that encode plant EPSPS polypeptides that comprise G102A and at least one or more amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid position set forth in SEQ ID NO:1 and wherein the plant EPSPS polypeptide comprises a sequence that is at least 90% identical to SEQ ID NO:2.

The EPSPS polypeptides and active variants and fragments thereof disclosed herein may have improved catalytic capacity in the presence of glyphosate when compared to previously identified EPSPS polypeptides. The parameter that best indicates the fitness of this trait in vivo is k_{cat}/K_{M}*K_{I}. The EPSPS polypeptides disclosed herein can have an increased k_{cat}/K_{M}*K_{I}, when compared to previously known EPSPS enzymes. By “increase” is intended any statistically significant increase when compared to an appropriate control. In some embodiments, an appropriate control is a previously known EPSPS sequence, such as that set forth in SEQ ID NO:2 (maize), SEQ ID NO:22 (rice), SEQ ID NO:23 (sorghum), SEQ ID NO:24 (sunflower), SEQ ID NO:20 or 21 (soybean), SEQ ID NO:25 (wheat), SEQ ID NO:26 (Brassica rapa), SEQ ID NO:27 (tomato), or SEQ ID NO:28 (potato). In some embodiments, the increase in the k_{cat}/K_{M}*K_{I} when compared to these native sequences can comprise about a 150, 200, 250, 300, 350, 400, 450, 500, 550, 600, 650, 700, 750, 800-fold or greater increase. In still further embodiments, k_{cat}/K_{M}*K_{I} may include, for example, a k_{cat}/K_{M}*K_{I} of more than about 2000, 2500, 3000, 3500, 4000, 4500, 5000, 5500, 6000, 6500, 7000, 7500, 8000, 8500, 9000, 9500, 10000, or more. The k_{cat}/K_{M}*K_{I} for the wild-type maize EPSPS is 11.8, while the k_{cat}/K_{M}*K_{I} of an EPSPS enzyme comprising 1031, 107S, and 445G is 2254.

As used herein, an “isolated” or “purified” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For purposes of this disclosure, “isolated” or “recombinant” when used to refer to nucleic acid molecules excludes isolated unmodified chromosomes. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein. When the polypeptide of the disclosure or a biologically active portion thereof is recombinantly produced, optimally culture medium represents less than about 30%, 20%, 10%, 5%, or 1% (by dry weight) of chemical precursors or non-protein-of-interest chemicals.

As used herein, a “recombinant” polynucleotide comprises a combination of two or more chemically linked nucleic acid segments which are not found directly joined in nature. By “directly joined” is intended the two nucleic acid segments are immediately adjacent and joined to one another by a chemical linkage. In specific embodiments, the recombinant polynucleotide comprises a polynucleotide of interest or active variant or fragment thereof such that an additional chemically linked nucleic acid segment is located either 5′, 3′ or internal to the polynucleotide of interest. Alternatively, the chemically-linked nucleic acid segment of the recombinant polynucleotide can be formed by the deletion of a sequence. The additional chemically linked nucleic acid segment or the sequence deleted to join the linked nucleic acid segments can be of any length, including for example, 1, 2, 3, 4, 5, 6, 7, 8, 9, 10, 15, 20 or greater nucleotides. Various methods for making such recombinant polynucleotides are disclosed herein, including, for example, by chemical synthesis or by the manipulation of isolated segments of polynucleotides by genetic engineering techniques. In specific embodiments, the recombinant polynucleotide can comprise a recombinant DNA sequence or a recombinant RNA sequence.

A “recombinant polypeptide” comprises a combination of two or more chemically linked amino acid segments which are not found directly joined in nature. In specific embodiments, the recombinant polypeptide comprises an additional chemically linked amino acid segment that is located either at the N-terminal, C-terminal or internal to the recombinant polypeptide. Alternatively, the chemically-linked amino acid segment of the recombinant polypeptide can be formed by deletion of at least one amino acid. The additional chemically linked amino acid segment or the deleted chemically linked amino acid segment can be of any length, including for example, 1, 2, 3, 4, 5, 6, 7, 8, 9, 10, 15, 20 or amino acids.

### B. Active Fragments and Variants of EPSPS Sequences

Methods and compositions are provided which employ polynucleotides and polypeptides having EPSPS activity. Moreover, any given variant or fragment of an EPSPS sequence may further comprise an improved catalytic capacity in the presence of the inhibitor glyphosate when compared to an appropriate control.

i. Polynucleotide and Polypeptide Fragments

Fragments and variants of the EPSPS polynucleotides and polypeptides provided herein are also encompassed by the present disclosure. By “fragment” is intended a portion of the polynucleotide or a portion of the amino acid sequence and hence protein encoded thereby. Fragments of a polynucleotide may encode protein fragments that retain EPSPS activity, and in specific embodiments, can further comprise an improved property such as improved catalytic capacity in the presence of glyphosate. Alternatively, fragments of a polynucleotide that are useful as hybridization probes or PCR primers generally do not encode fragment proteins retaining biological activity. In specific embodiments, a fragment of a recombinant polynucleotide or a recombinant polynucleotide construct comprises at least one junction of the two or more chemically linked or operably linked nucleic acid segments which are not found directly joined in nature. Thus, fragments of a nucleotide sequence may range from at least about 20 nucleotides, about 50 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1000 nucleotides, about 1100 nucleotides, about 1200 nucleotides, about 1300 nucleotides, and up to the full-length polynucleotide encoding the EPSPS polypeptides. A fragment of an EPSPS polynucleotide that encodes a biologically active portion of an EPSPS protein of the disclosure will encode at least 25, 50, 75, 100, 125, 150, 175, 200, 225, 250, 275, 300, 325, 350, 375, 400, or 425 amino acids, or up to the total number of amino acids present in a full-length EPSPS polypeptide.

Thus, a fragment of an EPSPS polynucleotide may encode a biologically active portion of an EPSPS polypeptide, or it may be a fragment that can be used as a hybridization probe or PCR primer using methods disclosed below. A biologically active portion of an EPSPS polypeptide can be prepared by isolating a portion of one of the EPSPS polynucleotides, expressing the encoded portion of the EPSPS polypeptides (e.g., by recombinant expression in vitro), and assessing the activity of the EPSPS portion of the EPSPS protein. Polynucleotides that are fragments of a EPSPS nucleotide sequence comprise at least 20, 50, 100, 200, 300, 400, 500, 600, 700, 800, 900, 1000, 1100, 1200, or 1300 contiguous nucleotides, or up to the number of nucleotides present in a full-length EPSPS polynucleotide disclosed herein.

Fragments of a polypeptide may encode protein fragments that retain EPSPS activity, and in specific embodiments, can further comprise an improved catalytic capacity in the presence of glyphosate when compared to an appropriate control. A fragment of a EPSPS polypeptide disclosed herein will encode at least 25, 50, 75, 100, 125, 150, 175, 200, 225, 250, 275, 300, 325, 350, 375, 400, or 425 contiguous amino acids, or up to the total number of amino acids present in a full-length EPSPS polypeptide. In specific embodiments, such polypeptide fragments are active fragments, and in still other embodiments, the polypeptide fragment comprises a recombinant polypeptide fragment. As used herein, a fragment of a recombinant polypeptide comprises at least one of a combination of two or more chemically linked amino acid segments which are not found directly joined in nature.

ii. Polynucleotide and Polypeptide Variants

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity, that is, have EPSPS activity. Moreover, any given variant or fragment may further comprise an improved specificity for glyphosate when compared to an appropriate control resulting in decreased non-specific acetylation of, e.g. an amino acid such as aspartate. Such variants may result from, for example, genetic polymorphism or from human manipulation.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the EPSPS polypeptides provided herein. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis or gene synthesis but which still encode an EPSPS polypeptide.

Biologically active variants of an EPSPS polypeptide disclosed herein (and the polynucleotide encoding the same) will have at least about 85%, 90%, 91%, 92%, 93%, 93.5%, 94%, 94.5%, 95%, 95.5%, 96%, 96.5%, 97%, 97.5%, 98%, 98.5%, 99%, 99.5%, or more sequence identity to the polypeptide of any one of SEQ ID NOS:1, 2, 4, 5, 6, 7, 8, 9, 10, 11, 12, 13, 14, 15, 16, 17, 18, 19, 20, 21, 22, 23, 24, 25, 26, 27, and 28 as determined by sequence alignment programs and parameters described elsewhere herein.

The EPSPS polypeptide and the active variants and fragments thereof may be altered in various ways including amino acid substitutions, deletions, truncations, and insertions. Methods for such manipulations are generally known in the art. For example, amino acid sequence variants and fragments of the EPSPS proteins can be prepared by mutations in the DNA. Methods for mutagenesis and polynucleotide alterations are well known in the art. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The mutations that will be made in the DNA encoding the variant must not place the sequence out of reading frame and optimally will not create complementary regions that could produce secondary mRNA structure. See, EP Patent Application Publication No. 75,444.

### C. Sequence Comparisons

The following terms are used to describe the sequence relationships between two or more polynucleotides or polypeptides.

As used herein, “reference sequence” is a predetermined sequence used as a basis for sequence comparison. A reference sequence may be a subset or the entirety of a specified sequence; for example, as a segment of a full-length cDNA or gene sequence, or the complete cDNA or gene sequence or protein sequence.

As used herein, “comparison window” makes reference to a contiguous and specified segment of a polypeptide sequence, wherein the polypeptide sequence in the comparison window may comprise additions or deletions (i.e., gaps) compared to the reference sequence (which does not comprise additions or deletions) for optimal alignment of the two polypeptides. Generally, the comparison window is at least 5, 10, 15, or 20 contiguous amino acids in length, or it can be 30, 40, 50, 100, or longer. Those of skill in the art understand that to avoid a high similarity to a reference sequence due to inclusion of gaps in the polypeptide sequence a gap penalty is typically introduced and is subtracted from the number of matches.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTP for proteins) can be used. Alignment may also be performed manually by inspection.

### D. Plants and Other Host Cells of Interest

Further provided are engineered host cells that are transduced (transformed or transfected) with one or more EPSPS sequences or active variants or fragments thereof. The EPSPS polypeptides or variants and fragments thereof can be expressed in any organism, including in non-animal cells such as plants, yeast, fungi, bacteria and the like. Details regarding non-animal cell culture can be found in Payne et al. (1992) Plant Cell and Tissue Culture in Liquid Systems, John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin, Heidelberg, N.Y.); and Atlas and Parks (eds.) The Handbook of Microbiological Media (1993) CRC Press, Boca Raton, Fla.

Plants, plant cells, plant parts and seeds, and grain having the EPSPS sequences disclosed herein are also provided. In specific embodiments, the plants and/or plant parts have stably incorporated at least one heterologous EPSPS polypeptide disclosed herein or an active variant or fragment thereof. In addition, the plants or organism of interest can comprise multiple EPSPS polynucleotides (i.e., at least 1, 2, 3, 4, 5, 6 or more).

In specific embodiments, the heterologous plant EPSPS polynucleotide in the plant or plant part is operably linked to a heterologous regulatory element, such as but not limited to a constitutive, tissue-preferred, or other promoter for expression in plants or a constitutive enhancer.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

The EPSPS sequences and active variants and fragments thereof disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, conifers, turf grasses (including cool seasonal grasses and warm seasonal grasses).

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing that which is disclosed include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true first such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus. In specific embodiments, plants of the present disclosure are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

Additional host cells of interest can be a eukaryotic cell, an animal cell, a protoplast, a tissue culture cell, prokaryotic cell, a bacterial cell, such as E. coli, B. subtilis, Streptomyces, Salmonella typhimurium, a gram positive bacteria, a purple bacteria, a green sulfur bacteria, a green non-sulfur bacteria, a cyanobacteria, a spirochetes, a thermatogale, a flavobacteria, bacteroides; a fungal cell, such as Saccharomyces cerevisiae, Pichia pastoris, and Neurospora crassa; an insect cell such as Drosophila and Spodoptera frugiperda; a mammalian cell such as CHO, COS, BHK, HEK 293 or Bowes melanoma, archaebacteria (i.e., Korarchaeota, Thermoproteus, Pyrodictium, Thermococcales, Methanogens, Archaeoglobus, and extreme Halophiles) and others.

For example, in some embodiments, glyphosate tolerant maize plants are provided, in which the glyphosate tolerant maize plants express an endogenous EPSPS polypeptide that has G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid position set forth in SEQ ID NO:1 and wherein the endogenous plant EPSPS gene encodes a polypeptide comprising a sequence that is at least 90% identical to SEQ ID NO:2.

Further, the glyphosate tolerant Brassica rapa plant may express an EPSPS polypeptide that has G102A and at least two, at least three, or at least four of the amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid position set forth in SEQ ID NO:1 and wherein the plant EPSPS gene encodes a polypeptide comprising a sequence that is at least 90% identical to SEQ ID NO:26.

### E. Polynucleotide Constructs

The use of the term “polynucleotide” is not intended to limit a polynucleotide of the disclosure to a polynucleotide comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the disclosure also encompass all forms of sequences including, but not limited to, single-stranded forms, double-stranded forms, hairpins, stem-and-loop structures, and the like.

For example, a polynucleotide construct may be a recombinant DNA construct. A “recombinant DNA construct” comprises two or more operably linked DNA segments which are not found operably linked in nature. Non-limiting examples of recombinant DNA constructs include a polynucleotide of interest or active variant or fragment thereof operably linked to heterologous sequences which aid in the expression, autologous replication, and/or genomic insertion of the sequence of interest. Such heterologous and operably linked sequences include, for example, promoters, termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The EPSPS polynucleotides disclosed herein can be provided in expression cassettes for expression in the plant of interest or any organism of interest. The cassette can include 5′ and 3′ regulatory sequences operably linked to an EPSPS polynucleotide or active variant or fragment thereof. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the EPSPS polynucleotide or active variant or fragment thereof to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a EPSPS polynucleotide or active variant or fragment thereof, and a transcriptional and translational termination region (i.e., termination region) functional in plants. The regulatory regions (i.e., promoters, transcriptional regulatory regions, and translational termination regions) and/or the EPSPS polynucleotide or active variant or fragment thereof may be native/analogous to the host cell or to each other. Alternatively, the regulatory regions and/or the EPSPS polynucleotide of or active variant or fragment thereof may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region or active variant or fragment thereof, may be native with the plant host, or may be derived from another source (i.e., foreign or heterologous) to the promoter, the EPSPS polynucleotide or active fragment or variant thereof, the plant host, or any combination thereof.

The expression cassettes may additionally contain 5′ leader sequences. Such leader sequences can act to enhance translation. Translation leaders are known in the art and include viral translational leader sequences.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used to express the various EPSPS sequences disclosed herein, including the native promoter of the polynucleotide sequence of interest. The promoters can be selected based on the desired outcome. Such promoters include, for example, constitutive, inducible, tissue-preferred, or other promoters for expression in plants or in any organism of interest.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Synthetic promoters can be used to express EPSPS sequences or biologically active variants and fragments thereof. Synthetic promoters include for example a combination of one or more heterologous regulatory elements.

In another aspect, the EPSPS sequences disclosed herein or active variants or fragments thereof can also be used as a selectable marker gene. In this embodiment, the presence of the EPSPS polynucleotide in a cell or organism confers upon the cell or organism the detectable phenotypic trait of glyphosate resistance, thereby allowing one to select for cells or organisms that have been transformed with a gene of interest linked to the EPSPS polynucleotide. Thus, for example, the EPSPS polynucleotide can be introduced into a nucleic acid construct, e.g., a vector, thereby allowing for the identification of a host (e.g., a cell or transgenic plant) containing the nucleic acid construct by growing the host in the presence of glyphosate and selecting for the ability to survive and/or grow at a rate that is discernibly greater than a host lacking the nucleic acid construct would survive or grow. An EPSPS polynucleotide can be used as a selectable marker in a wide variety of hosts that are sensitive to glyphosate, including plants, most bacteria (including E. coli), actinomycetes, yeasts, algae and fungi.

In specific embodiments, the EPSPS polypeptides and active variants and fragments thereof, and polynucleotides encoding the same, further comprise a chloroplast transit peptide. As used herein, the term “chloroplast transit peptide” will be abbreviated “CTP” and refers to the N-terminal portion of a chloroplast precursor protein that directs the latter into chloroplasts and is subsequently cleaved off by the chloroplast processing protease. When a CTP is operably linked to the N-terminus of a polypeptide, the polypeptide is translocated into the chloroplast. Removal of the CTP from a native protein reduces or abolishes the ability of the native protein from being transported into the chloroplast. An operably linked chloroplast transit peptide is found at the N-terminus of the protein to be targeted to the chloroplast and is located upstream and immediately adjacent to the transit peptide cleavage site that separates the transit peptide from the mature protein to be targeted to the chloroplast.

The term “chloroplast transit peptide cleavage site” refers to a site between two amino acids in a chloroplast-targeting sequence at which the chloroplast processing protease acts. Chloroplast transit peptides target the desired protein to the chloroplast and can facilitate the proteins translocation into the organelle. This is accompanied by the cleavage of the transit peptide from the mature polypeptide or protein at the appropriate transit peptide cleavage site by a chloroplast processing protease, native to the chloroplast. Accordingly, a chloroplast transit peptide further comprises a suitable cleavage site for the correct processing of the pre-protein to the mature polypeptide contained within the chloroplast.

As used herein, a “heterologous” CTP comprises a transit peptide sequence which is foreign to the polypeptide it is operably linked to. Such heterologous chloroplast transit peptides are known, including but not limited to those derived from Pisum (JP 1986224990; E00977), carrot (Luo et al. (1997) Plant Mol. Biol., 33 (4), 709-722 (Z33383), Nicotiana (Bowler et al., EP 0359617; A09029), Oryza (de Pater et al. (1990) Plant Mol. Biol., 15 (3), 399-406 (X51911), as well as synthetic sequences such as those provided in EP 0189707; U.S. Pat. Nos. 5,728,925; 5,717,084 (A10396 and A10398). In one embodiment, the heterologous chloroplast transit peptide is from the ribulose-1,5-bisphosphate carboxylase

(Rubisco) small subunit precursor protein isolated from any plant. The Rubisco small subunit is well characterized from a variety of plants and the transit peptide from any of them will be suitable for use disclosed herein. See for example, Physcomitrella (Quatrano et al., AW599738); Lotus (Poulsen et al., AW428760); Citrullus (J. S. Shin, A1563240); Nicotiana (Appleby et al. (1997) Heredity 79(6), 557-563); alfalfa (Khoudi et al. (1997) Gene, 197(1/2), 343-351); potato and tomato (Fritz et al. (1993) Gene, 137(2), 271-4); wheat (Galili et al. (1991) Theor. Appl. Genet. 81(1), 98-104); and rice (Xie et al. (1987) Sci. Sin., Ser. B (Engl. Ed.), 30(7), 706-19). For example, transit peptides may be derived from the Rubisco small subunit isolated from plants including but not limited to, soybean, rapeseed, sunflower, cotton, corn, tobacco, alfalfa, wheat, barley, oats, sorghum, rice, Arabidopsis, sugar beet, sugar cane, canola, millet, beans, peas, rye, flax, and forage grasses. Preferred for use in the present disclosure is the Rubisco small subunit precursor protein from, for example, Arabidopsis or tobacco. Such transit peptides are well known in the art and include, but are not limited to, the transit peptide for the acyl carrier protein, the small subunit of RUBISCO, plant EPSP synthase and Helianthus annuus (see Lebrun et al. U.S. Pat. No. 5,510,417), Zea mays Brittle-1 chloroplast transit peptide (Nelson et al. Plant Physiol. 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. In addition, chimeric chloroplast transit peptides are known in the art, such as the Optimized Transit Peptide (see, U.S. Pat. No. 5,510,471). Additional chloroplast transit peptides have been described previously in U.S. Pat. Nos. 5,717,084; 5,728,925 and the TraP14, Trap24, TraP23 transit peptides disclosed in US20130217577. One skilled in the art will readily appreciate the many options available in expressing a product to a particular organelle.

### F. Stacking Other Traits of Interest

In some embodiments, the EPSPS polynucleotides or active variants and fragments thereof disclosed herein are engineered into a molecular stack. Thus, the various host cells, plants, plant cells and seeds disclosed herein can further comprise one or more traits of interest, and in more specific embodiments, the host cell, plant, plant part or plant cell is stacked with any combination of polynucleotide sequences of interest in order to create plants with a desired combination of traits. As used herein, the term “stacked” includes having the multiple traits present in the same plant or organism of interest. In one non-limiting example, “stacked traits” comprise a molecular stack where the sequences are physically adjacent to each other. A trait, as used herein, refers to the phenotype derived from a particular sequence or groups of sequences. In one embodiment, the molecular stack comprises at least one additional polynucleotide that also confers tolerance to at least one sequence that confers tolerance to glyphosate by the same and/or different mechanism and/or at least one additional polynucleotide that confers tolerance to a second herbicide.

Thus, in one embodiment, the host cells, plants, plant cells or plant part having the EPSPS polynucleotide or active variants or fragments thereof disclosed herein is stacked with at least one other EPSPS sequence. Such EPSPS sequence include the EPSPS sequence and variants and fragment thereof disclosed herein, as well as other EPSPS sequences, which include but are not limited to, the EPSPS sequences set forth in WO02/36782, US Publication 2004/0082770 and WO 2005/012515, U.S. Pat. Nos. 7,462,481, 7,405,074, each of which is herein incorporated by reference.

The mechanism of glyphosate tolerance produced by the EPSPS sequences disclosed herein may be combined with other modes of herbicide resistance to provide host cells, plants, plant explants and plant cells that are tolerant to glyphosate and one or more other herbicides. For instance, the mechanism of glyphosate tolerance conferred by EPSPS may be combined with other modes of glyphosate tolerance known in the art. In other embodiments, the plant or plant cell or plant part having the EPSPS sequence or an active variant or fragment thereof may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glutamine synthetase (GS); glufosinate herbicides; herbicides which target the protox enzyme (also referred to as “protox inhibitors”).

The plant or plant cell or plant part having the EPSPS sequence or an active variant or fragment thereof can also be combined with at least one other trait to produce plants that further comprise a variety of desired trait combinations. For instance, the plant or plant cell or plant part having the EPSPS sequence or an active variant or fragment thereof may be stacked with polynucleotides encoding polypeptides having pesticidal and/or insecticidal activity, or a plant or plant cell or plant part having the EPSPS sequence or an active variant or fragment thereof may be combined with a plant disease resistance gene.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Any plant having at EPSPS sequence disclosed herein or an active variant or fragment thereof can be used to make a food or a feed product. Such methods comprise obtaining a plant, explant, seed, plant cell, or cell comprising the EPSPS sequence or active variant or fragment thereof and processing the plant, explant, seed, plant cell, or cell to produce a food or feed product.

### II. Methods of Use

### A. Methods of Generating Glyphosate Tolerant Plants

The terms “glyphosate tolerance” and “glyphosate resistance” are used interchangeably herein.

i. Introducing

Various methods can be used to introduce a sequence of interest into a host cell, plant or plant part. “Introducing” is intended to mean presenting to the host cell, plant, plant cell or plant part the polynucleotide or polypeptide in such a manner that the sequence gains access to the interior of a cell of the plant or organism. The methods of the disclosure do not depend on a particular method for introducing a sequence into an organism or a plant or plant part, only that the polynucleotide or polypeptides gains access to the interior of at least one cell of the organism or the plant. Methods for introducing polynucleotide or polypeptides into various organisms, including plants, are known in the art including, but not limited to, stable transformation methods, transient transformation methods, and virus-mediated methods.

“Stable transformation” is intended to mean that the nucleotide construct introduced into a plant integrates into the genome of the plant or organism of interest and is capable of being inherited by the progeny thereof. “Transient transformation” is intended to mean that a polynucleotide is introduced into the plant or organism of interest and does not integrate into the genome of the plant or organism or a polypeptide is introduced into a plant or organism.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the EPSPS sequences or active variants or fragments thereof can be provided to a plant using a variety of transient transformation methods. Such transient transformation methods include, but are not limited to, the introduction of the EPSPS protein or active variants and fragments thereof directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

In other embodiments, the EPSPS polynucleotide disclosed herein or active variants and fragments thereof may be introduced into plants by contacting plants with a virus or viral nucleic acids. Generally, such methods involve incorporating a nucleotide construct of the disclosure within a DNA or RNA molecule. It is recognized that the EPSPS sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide disclosed herein can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome.

Transformed plant cells which are derived by plant transformation techniques, including those discussed above, can be cultured to regenerate a whole plant which possesses the transformed genotype (i.e., a EPSPS polynucleotide), and thus the desired phenotype, such as acquired resistance (i.e., tolerance) to glyphosate or a glyphosate analog. For transformation and regeneration of maize see, Gordon-Kamm et al., The Plant Cell, 2:603-618 (1990). Plant regeneration from cultured protoplasts is described in Evans et al. (1983) Protoplasts Isolation and Culture, Handbook of Plant Cell Culture, pp 124-176, Macmillan Publishing Company, New York; and Binding (1985) Regeneration of Plants, Plant Protoplasts pp 21-73, CRC Press, Boca Raton. Regeneration can also be obtained from plant callus, explants, organs, or parts thereof. Such regeneration techniques are described generally in Klee et al. (1987) Ann Rev of Plant Phys 38:467.

One of skill will recognize that after the expression cassette containing the EPSPS gene is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included, provided that these parts comprise cells comprising the EPSPS nucleic acid. Progeny and variants, and mutants of the regenerated plants are also included, provided that these parts comprise the introduced nucleic acid sequences.

In one embodiment, a homozygous transgenic plant can be obtained by sexually mating (selfing) a heterozygous transgenic plant that contains a single added heterologous nucleic acid, germinating some of the seed produced and analyzing the resulting plants produced for altered cell division relative to a control plant (i.e., native, non-transgenic). Back-crossing to a parental plant and out-crossing with a non-transgenic plant are also contemplated.

Animal and lower eukaryotic (e.g., yeast) host cells are competent or rendered competent for transfection by various means. There are several well-known methods of introducing DNA into animal cells. These methods include: calcium phosphate precipitation; fusion of the recipient cells with bacterial protoplasts containing the DNA; treatment of the recipient cells with liposomes containing the DNA; DEAE dextran; electroporation; biolistics; and micro-injection of the DNA directly into the cells.

ii. Modifying

In general, methods to modify or alter the host genomic DNA are available. For example, a pre-existing or endogenous EPSPS sequence in a host plant can be modified or altered in a site-specific fashion using one or more site-specific engineering systems. This includes altering the host DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The CRISPR/Cas system allows targeted cleavage of genomic DNA guided by a customizable small noncoding RNA.

For instance, an endogenous plant EPSPS gene in a plant cell may be modified to encode a glyphosate tolerant EPSPS protein that comprises G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the endogenous plant EPSPS gene encodes a polypeptide comprising a sequence that is at least 90% identical to SEQ ID NO:2. A glyphosate tolerant plant may be grown from the plant cell. The modified endogenous plant EPSPS gene may encode a glyphosate tolerant EPSPS protein that comprises G102A and at least two, at least three, or at least four of the amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid position set forth in SEQ ID NO:1 and wherein the endogenous plant EPSPS gene encodes a polypeptide comprising a sequence that is at least 90% identical to SEQ ID NO:2. The modified endogenous plant EPSPS gene may encode a glyphosate tolerant EPSPS protein that comprises: (a) A4W, H54M, L98C, G102A, K173R, I208L, K243E, E3025, T361S, E391P, D402G, A416G, V438R, S440R, T441Q, and F442V; (b) A2R, A4W, A72Q, K84R, L98C, G102A, I208L, T279A, E3025, T361S, E391G, D402G, A416G, V438R, and T441Q; or (c) A2R, A4W, K84R, L98C, G102A, I208L, K243E, E391P, and D402G. The modified endogenous plant EPSPS gene may encode a glyphosate tolerant EPSPS protein that comprises the plant EPSPS polypeptide set forth in One of SEQ ID NOS: 3-12 and 45-59.

The endogenous plant EPSPS gene may be modified by a CRISPR/Cas guide RNA-mediated system, a Zn-finger nuclease-mediated system, a meganuclease-mediated system, an oligonucleobase-mediated system, or any gene modification system known to one of ordinary skill in the art.

Moreover, for the purposes herein, an endogenous plant EPSPS gene includes coding DNA and genomic DNA within and surrounding the coding DNA, such as for example, the promoter, intron, and terminator sequences.

In some embodiments, the CRISPR/Cas guide RNA-mediated system is used to modify the endogenous plant EPSPS gene. CRISPRs are arrays of clustered, regularly interspaced, short palindromic repeats within the bacterial genome. The recent discovery of CRISPR-associated protein 9 nuclease (Cas9) from Streptococcus pyogenes presents the possibility of introducing mutations into a native gene (Sander and Joung, 2014). To introduce double strand breaks into the target gene, Cas9 is guided to the target gene DNA by normal base-pairing with an engineered RNA. Following double-strand break, the desired mutation(s) in EPSPS can be introduced from an engineered template through the homology-directed repair process. EPSPS coded by modified genes will be under the control of the native promoter. Thus, all tissues will express the enzyme according to their native spatial and temporal program, a condition that may confer an advantage over transgenic expression in providing appropriate catalytic capacity.

As used herein, the term “guide polynucleotide”, refers to a polynucleotide sequence that can form a complex with a Cas endonuclease and enables the Cas endonuclease to recognize and optionally cleave a DNA target site. The guide polynucleotide can include a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. In some embodiment of this disclosure, the guide polynucleotide does not solely comprise ribonucleic acids (RNAs). A guide polynucleotide that solely comprises ribonucleic acids is also referred to as a “guide RNA”.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In one embodiment, the nucleotide sequence linking the crNucleotide and the tracrNucleotide of a single guide polynucleotide can comprise a tetraloop sequence, such as, but not limiting to a GAAA tetraloop sequence.

In one embodiment the guide polynucleotide can be introduce into the plant cell directly using any method known to one skilled in the art, such as for example, but not limited to, particle bombardment or topical applications.

When the guide polynucleotide comprises solely of RNA sequences (also referred to as “guide RNA”) it can be introduced indirectly by introducing a recombinant DNA molecule comprising the corresponding guide DNA sequence operably linked to a plant specific promoter that is capable of transcribing the guide polynucleotide in said plant cell. The term “corresponding guide DNA” refers to a DNA molecule that is identical to the RNA molecule but has a “T” substituted for each “U” of the RNA molecule.

In some embodiments, the guide polynucleotide is introduced via particle bombardment or Agrobacterium transformation of a recombinant DNA construct comprising the corresponding guide DNA operably linked to a plant U6 polymerase III promoter.

The terms “target site”, “target sequence”, “target DNA”, “target locus”, “genomic target site”, “genomic target sequence”, and “genomic target locus” are used interchangeably herein and refer to a polynucleotide sequence in the genome (including chloroplastic and mitochondrial DNA) of a cell at which a double-strand break is induced in the cell genome by a Cas endonuclease. The target site can be an endogenous site in the genome of a cell or organism, or alternatively, the target site can be heterologous to the cell or organism and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a cell or organism and is at the endogenous or native position of that target sequence in the genome of a cell or organism. Cells include, but are not limited to animal, bacterial, fungal, insect, yeast, and plant cells as well as plants and seeds produced by the methods described herein.

In one embodiments, the target site, in association with the particular gene editing system that is being used, can be similar to a DNA recognition site or target site that is specifically recognized and/or bound by a double-strand break inducing agent, such as but not limited to a Zinc Finger endonuclease, a meganuclease, or a TALEN endonuclease.

An “artificial target site” or “artificial target sequence” are used interchangeably herein and refer to a target sequence that has been introduced into the genome of a cell or organism, such as but not limiting to a plant or yeast. Such an artificial target sequence can be identical in sequence to an endogenous or native target sequence in the genome of a cell but be located in a different position (i.e., a non-endogenous or non-native position) in the genome of a cell or organism.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Polynucleotide constructs that provide a guide RNA which targets an endogenous EPSPS gene of a plant cell are provided herein. The polynucleotide construct may further comprise one or more polynucleotide modification templates to generate a modified endogenous EPSPS gene that encodes a plant EPSPS polypeptide that comprises G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the endogenous plant EPSPS gene encodes a polypeptide comprising a sequence that is at least 90% identical to SEQ ID NO:2. The modified endogenous EPSPS gene may encode a plant EPSPS polypeptide that comprises G102A and at least two, at least three, or at least four amino acid mutations selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid position corresponds to the amino acid mutation position set forth in SEQ ID NO:1 and wherein the endogenous plant EPSPS gene encodes a polypeptide comprising a sequence that is at least 90% identical to SEQ ID NO:2. The modified endogenous EPSPS gene may encode a plant EPSPS polypeptide that comprises: (a) A4W, H54M, L98C, G102A, K173R, I208L, K243E, E3025, T361S, E391P, D402G, A416G, V438R, S440R, T441Q, and F442V; (b) A2R, A4W, A72Q, K84R, L98C, G102A, I208L, T279A, E3025, T361S, E391G, D402G, A416G, V438R, and T441Q; or (c) A2R, A4W, K84R, L98C, G102A, I208L, K243E, E391P, and D402G. The modified endogenous EPSPS gene may encode a plant EPSPS polypeptide that has the amino acid sequence set forth in One of SEQ ID NOS: 3-12.

Methods for producing glyphosate tolerant plants are provided herein in which a guide RNA, one or more polynucleotide modification templates, and one or more Cas endonucleases are provided to a plant cell. The Cas endonuclease(s) introduces a double strand break at an endogenous EPSPS gene in the plant cell, and the polynucleotide modification template(s) is used to generate a modified EPSPS gene that encodes a plant EPSPS polypeptide that comprises G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the endogenous plant EPSPS gene encodes a polypeptide comprising a sequence that is at least 90% identical to SEQ ID NO:2. A plant is obtained from the plant cell, and a glyphosate tolerant progeny plant that is void of the guide RNA and Cas endonuclease is generated.

B. Methods for Increasing Expression and/or Activity Level of at Least One EPSPS Sequence or an Active Variant or Fragment Thereof in a Host Cell of Interest, a Plant or Plant Part

Various methods are provided for the expression of an EPSPS sequence or active variant or fragment thereof in a host cell of interest. For example, the host cell of interest is transformed with the EPSPS sequence and the cells are cultured under conditions which allow for the expression of the EPSPS sequence. In some embodiments, the cells are harvested by centrifugation, disrupted by physical or chemical means, and the resulting crude extract retained for further purification. Microbial cells employed in the expression of proteins can be disrupted by any convenient method, including freeze-thaw cycling, sonication, mechanical disruption, or use of cell lysing agents, or other methods, which are well known to those skilled in the art.

### C. Method of Producing Crops and Controlling Weeds

Methods for controlling weeds in an area of cultivation, preventing the development or the appearance of herbicide resistant weeds in an area of cultivation, producing a crop, and increasing crop safety are provided. The term “controlling,” and derivations thereof, for example, as in “controlling weeds” refers to one or more of inhibiting the growth, germination, reproduction, and/or proliferation of; and/or killing, removing, destroying, or otherwise diminishing the occurrence and/or activity of a weed.

As used herein, an “area of cultivation” comprises any region in which one desires to grow a plant. Such areas of cultivations include, but are not limited to, a field in which a plant is cultivated (such as a crop field, a sod field, a tree field, a managed forest, a field for culturing fruits and vegetables, etc.), a greenhouse, a growth chamber, etc.

As used herein, by “selectively controlled” it is intended that the majority of weeds in an area of cultivation are significantly damaged or killed, while if crop plants are also present in the field, the majority of the crop plants are not significantly damaged. Thus, a method is considered to selectively control weeds when at least 55%, 60%, 65%, 70%, 75%, 80%, 85%, 90%, 95%, or more of the weeds are significantly damaged or killed, while if crop plants are also present in the field, less than 10%, 5%, or 1% of the crop plants are significantly damaged or killed.

Methods provided comprise planting the area of cultivation with a plant having a EPSPS sequence or active variant or fragment thereof disclosed herein or transgenic seed derived therefrom, and in specific embodiments, applying to the crop, seed, weed or area of cultivation thereof an effective amount of a herbicide of interest. It is recognized that the herbicide can be applied before or after the crop is planted in the area of cultivation. Such herbicide applications can include an application of glyphosate.

Accordingly, the term “glyphosate” should be considered to include any herbicidally effective form of N-phosphonomethylglycine (including any salt thereof) and other forms which result in the production of the glyphosate anion in planta.

In specific methods, glyphosate is applied to the plants having the EPSPS sequence or active variant or fragment thereof or their area of cultivation. In specific embodiments, the glyphosate is in the form of a salt, such as, ammonium, isopropylammonium, potassium, sodium (including sesquisodium) or trimesium (alternatively named sulfosate). In still further embodiments, a mixture of a synergistically effective amount of a combination of glyphosate and an ALS inhibitor (such as a sulfonylurea) is applied to the plants or their area of cultivation.

Generally, the effective amount of herbicide applied to the field is sufficient to selectively control the weeds without significantly affecting the crop. In some embodiments, the effective amount of glyphosate applied is about 50 gram acid equivalent/acre to about 2000 gram acid equivalent/acre. It is important to note that it is not necessary for the crop to be totally insensitive to the herbicide, so long as the benefit derived from the inhibition of weeds outweighs any negative impact of the glyphosate or glyphosate analog on the crop or crop plant.

“Weed” as used herein refers to a plant which is not desirable in a particular area. Conversely, a “crop plant” as used herein refers to a plant which is desired in a particular area, such as, for example, a maize or soy plant. Thus, in some embodiments, a weed is a non-crop plant or a non-crop species, while in some embodiments, a weed is a crop species which is sought to be eliminated from a particular area, such as, for example, an inferior and/or non-transgenic soy plant in a field planted with a plant having the EPSPS sequence disclosed herein or an active variant or fragment thereof.

Accordingly, the current disclosure provides methods for selectively controlling weeds in a field containing a crop that involve planting the field with crop seeds or plants which are glyphosate-tolerant as a result of being transformed with a gene encoding a EPSPS disclosed herein or an active variant or fragment thereof, and applying to the crop and weeds in the field a sufficient amount of glyphosate to control the weeds without significantly affecting the crop.

Further provided are methods for controlling weeds in a field and preventing the emergence of glyphosate resistant weeds in a field containing a crop which involve planting the field with crop seeds or plants that are glyphosate tolerant as a result of being transformed with a gene encoding EPSPS and a gene encoding a polypeptide imparting glyphosate tolerance by another mechanism, such as, a glyphosate tolerant glyphosate-N-acetyltransferase and/or a glyphosate-tolerant glyphosate oxido-reductase and applying to the crop and the weeds in the field a sufficient amount of glyphosate to control the weeds without significantly affecting the crop. Various plants that can be used in this method are discussed in detail elsewhere herein.

In further embodiments, the current disclosure provides methods for controlling weeds in a field and preventing the emergence of herbicide resistant weeds in a field containing a crop which involve planting the field with crop seeds or plants that are glyphosate tolerant as a result of being transformed with a gene encoding EPSPS, a gene encoding a polypeptide imparting glyphosate tolerance by another mechanism, such as, a glyphosate tolerant glyphosate-N-acetyltransferase and/or a glyphosate oxido-reductase and a gene encoding a polypeptide imparting tolerance to an additional herbicide, such as, a mutated hydroxyphenylpyruvatedioxygenase, a sulfonylurea-tolerant acetolactate synthase, a sulfonylurea-tolerant acetohydroxy acid synthase, a sulfonamide-tolerant acetolactate synthase, a sulfonamide-tolerant acetohydroxy acid synthase, an imidazolinone-tolerant acetolactate synthase, an imidazolinone-tolerant acetohydroxy acid synthase, a phosphinothricin acetyl transferase and a mutated protoporphyrinogen oxidase and applying to the crop and the weeds in the field a sufficient amount of glyphosate and an additional herbicide, such as, a hydroxyphenylpyruvatedioxygenase inhibitor, sulfonamide, imidazolinone, bialaphos, phosphinothricin, azafenidin, butafenacil, sulfosate, glufosinate, and a protox inhibitor to control the weeds without significantly affecting the crop. Various plants and seeds that can be used in this method are discussed in detail elsewhere herein.

Further provided are methods for controlling weeds in a field and preventing the emergence of herbicide resistant weeds in a field containing a crop which involve planting the field with crop seeds or plants that are glyphosate tolerant as a result of being transformed with a gene encoding an EPSPS and a gene encoding a polypeptide imparting tolerance to an additional herbicide, such as, a mutated hydroxyphenylpyruvatedioxygenase, a sulfonamide-tolerant acetolactate synthase, a sulfonamide-tolerant acetohydroxy acid synthase, an imidazolinone-tolerant acetolactate synthase, an imidazolinone-tolerant acetohydroxy acid synthase, a phosphinothricin acetyl transferase and a mutated protoporphyrinogen oxidase and applying to the crop and the weeds in the field a sufficient amount of glyphosate and an additional herbicide, such as, a hydroxyphenylpyruvatedioxygenase inhibitor, sulfonamide, imidazolinone, bialaphos, phosphinothricin, azafenidin, butafenacil, sulfosate, glufosinate, and a protox inhibitor to control the weeds without significantly affecting the crop. Various plants and seeds that can be used in this method are discussed in detail elsewhere herein.

In some embodiments, a plant of the disclosure is not significantly damaged by treatment with a glyphosate herbicide applied to that plant at a dose equivalent to a rate of at least 0.5, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 110, 120, 150, 170, 200, 300, 400, 500, 600, 700, 800, 800, 1000, 2000, 3000, 4000, 5000, 5400 or more grams or ounces (1 ounce=29.57 ml) of active ingredient or commercial product or herbicide formulation per acre or per hectare, whereas an appropriate control plant is significantly damaged by the same glyphosate treatment.

### III. A Rapid Assay for Catalytic Efficiency of a Plurality of Enzyme Variants

One of the commercial applications of directed evolution is to desensitize an enzyme to inhibition by, for example, a herbicide. kcat, 1/KM, and KI are three dimensions that when multiplied are a measure of an enzyme's intrinsic capacity for catalysis in the presence of an inhibitor. The ideal values for the individual dimensions depend on substrate and inhibitor concentrations under the conditions of the application. When attempting to optimize those values by directed evolution, (kcat/KM)*KI can be an informative parameter for evaluating libraries of variants. However, evaluating (kcat/KM)*KI for hundreds of variants by substrate saturation analysis may not provide adequate throughput. A manipulation of the Michaelis-Menten equation that enables isolation of (kcat/KM)*KI on one side of the equation is presented herein. If substrate and enzyme concentrations are identical but velocity is measured at two different inhibitor concentrations (one of which can be 0), the data are sufficient to calculate (kcat/KM)*KI with just two rate measurements. The procedure has been validated by correlating values obtained with the rapid method with those obtained by substrate saturation kinetics.

The method includes (a) providing a plurality of enzyme variants; (b) providing the inhibitor; (c) providing the substrate; (d) performing a reaction involving the plurality of enzyme variants and the substrate, at no more than two different inhibitor concentrations; (e) measuring reaction rate at no more than two different inhibitor concentrations; and (f) calculating (kcat/KM)*KI of the plurality of enzyme variants. In some embodiments, one of the inhibitor concentrations is zero. In other embodiments, the substrate is at a concentration that is substantially similar to Michaelis-Menten constant (KM) of a parental enzyme for the enzyme variant. In still other embodiments, the enzyme is at a sufficient concentration to result in a substantially linear reaction rate at the two different inhibitor concentrations. In still other embodiments, one of the inhibitor concentrations is sufficient to result in at least about 50% inhibition. In still other embodiments, the assay is performed in a high-throughput system. In still other embodiments, the catalytic capacity in the presence of the inhibitor is estimated by obtaining a numerical value for (kcat/KM)*KI, wherein kcat is maximum enzyme turnover rate, KM is Michaelis-Menten constant and KI is inhibitor dissociation constant. In some embodiments, the substrate is PEP; the inhibitor is glyphosate; and the plurality of enzyme variants are EPSPS enzyme variants. In still other embodiments, the enzyme and the substrate concentrations are the same, at the two inhibitor concentrations.

### EXAMPLES

In the following Examples, unless otherwise stated, in which parts and percentages are by weight and degrees are Celsius. It should be understood that these Examples, while indicating embodiments of the invention, are given by way of illustration only. From the above discussion and these Examples, one skilled in the art, can make various changes and modifications of the invention to adapt it to various usages and conditions. Such modifications are also intended to fall within the scope of the appended embodiments.

### Example 1

### Fitness Parameter for Variants of EPSPS for Conferring Tolerance to Glyphosate

The fitness of a variant of EPSPS for conferring tolerance to glyphosate corresponds to the velocity of the enzyme-catalyzed reaction in the presence of glyphosate, as given by the Michaelis-Menten equation for competitive inhibition,

$${v}_{i} = \frac{{k}_{cat} [ E ] [ S ]}{{K}_{M} ( 1 + \frac{[ I ]}{{K}_{I}} ) + [ S ]}$$

where vᵢ is the initial reaction velocity, [E] is the enzyme concentration, [S] is the concentration of phosphoenolpyruvate (PEP), [I] is the concentration of glyphosate, k_{cat} is the reaction rate as a function of [E] at saturating [S] and KM is [S] at half maximal saturating [S]. A simplifying assumption is made that the non-competing substrate, shikimate-3-phosphate (S3P), is present at saturation, and therefore does not impact the equation. The parameter kcat/K_{M}*K_{I} has been used previously to indicate fitness, but that measure may not convey true kinetic fitness, especially if K_{I} is very high, exhibited by many variants described herein. The equation shows that velocity does not increase proportionately with increasing K_{I}. When K_{I} is higher than the inhibitor concentration, the reaction velocity can only increase a further 2-fold regardless of further increases in the value of K_{I}.

A more straight-forward, accurate and meaningful parameter is one in which one rate measurement is performed under presumed in vivo conditions, if known. To establish a set of conditions that mimic those under which the mutated enzyme needs to perform, the concentrations of PEP and S3P were set low (30 uM, subject to the sensitivity of assay conditions) to approach the presumed intracellular concentrations (˜15 uM, the approximate values of KM for both PEP and S3P). Glyphosate is included at 1 mM. The pH is set at 7.0 and ionic strength at 100 mM KCl, also to mimic known in vivo conditions. Ethylene glycol is present at 5% (v/v) to approximate the dielectric constant of intracellular fluid. The parameter is termed “enzyme turnover under application conditions”, which is herein shortened to “kcat gly”. The conventional formatting of the expression “k_{cat}” is not used, to avoid this term being characterized as a standard kinetic constant.

EPSPS activity was determined by quantifying the phosphate generated from the EPSPS reaction. Release of inorganic phosphate was coupled to its reaction with 2-amino-6-mercapto-7-methylpurine ribonucleoside (MESG), catalyzed by purine nucleoside phosphorylase, according to previously described protocols. To determine the kcat gly, enzyme preparations were normalized to 0.1 mg/ml and four- to six-microliter aliquots of normalized enzyme were added to the wells of a low UV-absorbing 96-well assay plate (Greiner UV-Star). Reactions were started with the addition of 2×147 μl of reaction mixture containing 25 mM Hepes, pH 7.0, 100 mM KCl, 5% ethylene glycol, 30 μM each of PEP and S3P, 0.15 mM MESG, 1.5 U/ml purine nucleoside phosphorylase (Sigma N8264) and 1 mM glyphosate. Absorbance was monitored at 4 sec intervals for 50 sec with a Spectramax plate reader (Molecular Devices). The maximal reaction rate obtainable from 6 time points was converted to uM/min using the extinction coefficient (11,200 M⁻¹ cm⁻¹) for the absorbance change that occurs with the conversion of MESG to 7-methyl-6-thioguanine. Division by the enzyme concentration (0.04 to 0.1 uM, depending on activity) yields “kcat gly” in units of min⁻¹.

### Example 2

### High Stringency Selection of Increasingly Fit Variants of EPSPS

Requiring an E. coli host expressing a shuffled variant of EPSPS to form a colony on minimal medium containing glyphosate is useful for screening large libraries of shuffled variants. Colonies that grow are picked and the cells are then grown in rich medium optimal for expression of the plasmid-encoded variant of EPSPS. The stringency of the selection can be increased by withholding the inducer and by reducing the copy number of the plasmid. To achieve the latter, the pET16b vector described previously was modified so that the origin of replication, ColE1, was replaced with the one found in pSC101, which typically generates ˜5 copies of the plasmid instead of ˜20. The improvement in stringency was assessed by plating E. coli cells transformed with plasmids expressing maize EPSPS variant. After 48 hrs., cells with the high copy plasmid grew well at glyphosate concentrations up to 200 mM, but in the low copy plasmid, the maize EPSPS variant could not support colony growth even in the absence of glyphosate. The most stringent selection medium was M9 agar containing 2% glucose, 0.1 mg/L polymyxin B nonapeptide, 1 mM betaine and 300 mM glyphosate.

### Example 3

### Maize EPSPS Variants with Increased Fitness in the Presence of Glyphosate

Single mutations that are neutral or beneficial in the context of the native maize EPSPS, Zm E1, Zm H6 and Zm C1 backbones were identified by performing saturation mutagenesis, as described previously. The mutations identified are shown in Table 1.

TABLE 1
Neutral or beneficial diversity identified
by performing saturation on EPSPS variants.

| | Native | | G102A | | E1 | | H6 | | D2 | | D2-67 | | F3 | |
|---|---|---|---|---|---|---|---|---|---|---|---|---|---|---|
| Pos | BB | DIV | BB | DIV | BB | DIV | BB | DIV | BB | DIV | BB | DIV | BB | DIV |
| 2 | A | P | | | | R | R | | R | L | R | | | |
| 3 | G | | | FS | | | | | | K V | K | | | |
| 4 | A | LNR | | | | PVW | W | | W | | W | | | |
| 6 | E | S | | IC | | | | | | G | | | | |
| 10 | Q | | | | | | | | | | | G | | |
| 13 | K | | | | | | | | | | | A | | |
| 18 | T | | | | | | | G | | | | | | |
| 19 | V | | | I | | | | | | | | | | |
| 20 | K | | | D | | | | | | | | | | |
| 36 | A | | | | | G | | G | | G | | | | |
| 38 | S | | | | | | | | | A | | | | |
| 39 | E | | | S | | G | | | | | | | | Y |
| 45 | D | | | | | | | | | | | | | L |
| 51 | E | | | L | | | | | | | | | | |
| 53 | V | | | | | | | I | | | | | | |
| 54 | H | | | EGS | | M | | E D | | L V | | G | | G |
| | | | | V | | | | N | | G | | | | |
| 61 | R | | | HT | | | | | | W | | Y | | E |
| 62 | T | | | | | | | | | V | | | | |
| 65 | L | | | | | | | V | | V | | V | | |
| 68 | E | | | | | | | | | | | | | G |
| 69 | A | | | M | | | | VTW | H | | H | | | |
| | | | | | | | | H | | | | | | |
| 70 | D | | | | | | | | | Y | | | | |
| 71 | K | | | | | | | P | | G | | | | |
| 72 | A | | | V | | GQ | Q | E V | | S K | | W, E | | K |
| 74 | K | | | | | | | V L | | | | | | |
| 76 | A | T | | | | | | V C | | C | | | | |
| 78 | V | L | | | | | | | | | | | | |
| 79 | V | | | | | | | | | RCW | | | | |
| 83 | G | | | | | | | | | D | | | | |
| 84 | K | | | | | R | R | | R | | R | | | |
| 86 | P | | | | | | | | | L | | | | |
| 87 | V | | | | | | | T | | | | M | | |
| 89 | D | | | | | | | F | | | | | | |
| 91 | K | | | | | | | G | | | | R | | |
| 92 | E | | | | | | | G | G | E | G | | | |
| 98 | L | | | V | | C | C | | C | | C | | C | |
| 101 | A | S | | | | | | | | | | | | |
| 102 | G | | A | A | A | | A | | A | | A | | A | |
| 103 | T | IAL | | | | | | | | | | | | |
| | | GV | | | | | | | | | | | | |
| 107 | P | GLQ | | | | | | | | | | | | |
| | | SW | | | | | | | | | | | | |
| | | A | | | | | | | | | | | | |
| 109 | T | | | | | | | | | V S | | | | |
| 113 | T | | | | | | | | | V | | | | |
| 118 | N | | | | | | | C | | | | | | |
| 124 | D | | | | | N | | | | | | | | |
| 127 | P | | | | | | | | | M | | | | |
| 143 | Q | | | | | | | | | G | | | | |
| 147 | D | | | | | | | | | | | R | | |
| 152 | L | | | C | | | | | | | | | | |
| 155 | D | | | AG | | | | | | | | | | |
| 156 | C | GY | | | | | | | | | | | | |
| 160 | R | | | | | | | | | | | | | G |
| 162 | N | | | | | | | | | | | T | | |
| 164 | I | | | | | | | R | | | | M | | |
| 168 | P | | | | | | | | | | | | | K |
| 172 | V | | | | | | | | | | | | | T |
| 173 | K | | | | | R | | | | | | | | |
| 177 | S | | | | | | | M | | Q | | G | | |
| 178 | I | | | | | | | | | R | | | | |
| 183 | L | | | | | | | | | | | | | C |
| 184 | S | | | | | | | | | | | | | T |
| 189 | A | | | | | | | | | | | | | S |
| 191 | V | | | I | | | | | | | | | | |
| 190 | A | | | | | S | | | | | | V | | G |
| 194 | L | M | | C | | | | | | | | | | V |
| 196 | D | | | | | | | E | | | | | | W |
| 197 | V | | | | | | | | | S | | | | |
| 201 | I | | | | | | | | | | | V | | |
| 202 | I | R | | | | | | | | | | | | |
| 208 | I | V | | | | LAS | L | H | L | V | L | G | | |
| | | | | | | REG | | | | | | | | |
| 216 | R | | | | | | | | | | | | | V |
| 217 | L | | | | | | | | | T | | | | |
| 219 | E | | | | | | | | | G | | | | |
| 220 | R | | | | | | | | | | | | | |
| 224 | K | R | | | | RQ | | | | | | G | | |
| 225 | A | | | | | | | | | | | G | | |
| 226 | E | | | | | | | | | G | | A | | Y |
| 229 | D | | | | | | | | | | | G | | |
| 230 | S | | | | | | | | | | | V | | |
| 232 | D | | | R | | | | | | | | G | | H |
| 233 | R | | | | | M | | | | | | H | | |
| 235 | Y | | | | | | | | | | | S | | |
| 238 | G | | | | | | | | | | | | | V |
| 239 | G | | | | | M | | | | | | M A | | |
| 240 | Q | | | | | | | | | | | | | V |
| 241 | K | A | | | | AV | | | | S | | R | | |
| 243 | K | | | | | | | | E | L R | E | R | | |
| 246 | K | G | | | | | | G | | R G | | V I | | |
| 247 | N | | | | | LQ | | | | | | RAT | | V |
| 248 | A | | | | | | | V | | | | | | |
| 249 | Y | V | | | | | | | | | | | | |
| 257 | A | | | | | | | | | | | G | | |
| 262 | A | | | | | | | | | | | G | | |
| 273 | V | | | | | | | | | | | | | A |
| 278 | T | VG | | | | | | | | | | | | |
| 279 | T | | | | | A | A | | | V | | R | | |
| 297 | K | | | | | | | SRD | | | | | | A |
| 298 | V | | | | | | | | | L | | | | |
| 302 | E | S | | | S | | S | | | | | | | |
| 308 | T | | | | | | | | | | | AS | | |
| 310 | P | V | | | | | | | | | | A | | |
| 311 | P | S | | | | | | | | | | VRE | | |
| | | | | | | | | | | | | FGS | | |
| | | | | | | | | | | | | AL | | |
| 313 | E | G | | | | | | G | | S | | | | |
| 314 | P | | | | | | | | | M | | | | |
| 315 | F | | | | | | | | | V R | | G | | |
| 316 | G | | | | | | | | | M | | | | F |
| 323 | I | | | | | | | | | | | | | E |
| 326 | N | | | | | | | | | T | | | | |
| 328 | N | C | | | | | | | | | | | | |
| 329 | | | | | | | | | | | | | | |
| 333 | V | | | | | | | | A | | A | | | A |
| 334 | A | | | | | | | | | | | G | | |
| 338 | A | S | | | | | | | | | | | | |
| 349 | A | | | | | | | | | M L | | N | | |
| 354 | A | | | | | | | | | G | G | | | |
| 361 | T | | | | | S | S | | | | | | S | |
| 366 | A | | | | | | | | | | | | | G |
| 368 | R | | | | | | | | | CME | | | | |
| 379 | E | | | | | | | | | D | | | | |
| 382 | P | | | | | | | D | | V G | | G | | |
| 388 | T | | | | | | | | | S | | | | |
| 391 | E | G | | | G | KPV | P | | P | E A | P | | | |
| 392 | K | | | | | | | | | | | G | | |
| 394 | N | | | | | Q | | | | A | | E | | |
| 397 | A | | | | | | | | | S | | | | |
| 402 | D | G | | | | | G | | G | | G | | G | |
| 416 | A | | | | | G | G | | | | | G | | |
| 419 | P | | | | | | | | | E R | | | | |
| 426 | G | | | S | | | | | | | | | | |
| 429 | R | | | | | | | | | H | | GV | | A |
| 432 | F | | | | | | | | | | | W | | |
| 434 | D | | | | | | | | | | | A | | |
| 437 | D | R | | | | | | | | G | | EYR | | S |
| | | | | | | | | | | | | G | | |
| 438 | V | R | | | R | | R | | | | | | | |
| 440 | S | | | | | ILR | | | | | | | | |
| 441 | T | | | | | AQ | Q | | | E G | | G | | |
| 442 | F | | | | | V | | | | | | | | |
| 445 | N | G | | | | | | | | | | | | |

Mutagenesis on native maize EPSPS and shuffled variants was performed. Amino acids are identified by the one-letter code. For variants other than native, neutral or beneficial is defined as having a value for kcat gly of >80% of the parental enzyme.

Approximately 30 substitutions were designed into each library. Assuming an average incorporation rate of six substitutions per variant, the theoretical size of such libraries is ˜7×10⁵. Libraries were screened using methods described previously and the enhanced method described in Example 2. The number of colony-forming units plated and screened was in the range of 50 to 100% of the theoretical size. Up to 200 colonies per library were picked for evaluation of the expressed variant. Proteins were purified as described previously in PCT/US2016/054399, incorporated herein by reference. Protein concentration was determined by measuring optical density at 280 nm. The extinction coefficient of native maize EPSPS (0.676 OD/mg/ml) was calculated by vNTI and used to convert OD280 to mg/ml. Kinetic parameters were determined as described in Example 1. Novel variants with fitness improved relative other variants are shown in Table 2.

G102A confers varying degrees of glyphosate insensitivity, depending on the amino acid sequence context. The additional methyl group has been shown to project into the active site, causing steric hindrance in the binding of glyphosate but also PEP in Class I EPSPS, but to interfere only with glyphosate binding in Class II EPSPS, such as CP4. K_{M} and K_{I} for G102A indicate reduced affinity for both glyphosate and PEP, as well as reduced k_{cat} relative to native maize EPSPS. Saturation mutagenesis in the G102A context enabled discovery of mutations that significantly ameliorated the undesired impact of the G102A mutation alone. The concerted effects of improved k_{cat}, K_{M} PEP and k_{cat}/K_{M} resulted in two-fold higher k_{cat} gly for the G102A-L98V, G102A-D155G and G102A-L194C variants relative to G102A alone, despite reductions in K_{I}. Further mutagenesis of G102A had the effect of further lowering the K_{M} for PEP while lowering K_{I} to a much lesser degree. The ability of the variant to discriminate between glyphosate and PEP is given by the value K_{I}/K_{M}, which increased from 5.1 with G102A to over 60 with several of the variants (Table 2).

TABLE 2
Kinetic parameters of variants of
maize EPSPS with enhanced fitness

| | k_{cat}, | | k_{cat}/ | | k_{cat}/ | kcat | K_{I}/ |
|---|---|---|---|---|---|---|---|
| Variant | min⁻¹ | K_{M}, uM | K_{M} | K_{I}, uM | K_{M}*K_{I} | gly | K_{M} |
| Zm native | 1464 | 15.7 | 93.6 | 0.13 | 11.8 | 0.0 | 0.008 |
| G102A | 612 | 315 | 1.93 | 1611 | 3176 | 12.2 | 5.11 |
| G102A- | 871 | 105 | 8.44 | 670 | 5627 | 30.4 | 6.40 |
| L098V | | | | | | | |
| G102A- | 718 | 177 | 4.06 | 940 | 3812 | 21.6 | 5.31 |
| D155G | | | | | | | |
| G102A- | 712 | 189 | 3.77 | 462 | 1740 | 22.3 | 2.44 |
| L194C | | | | | | | |
| G102A- | 640 | 178 | 3.60 | 906 | 3260 | 19.7 | 5.09 |
| H054E | | | | | | | |
| G102A- | 462 | 90.5 | 5.10 | 426 | 2174 | 18.1 | 4.71 |
| D232R | | | | | | | |
| Zm C1 | 495 | 24.6 | 20.1 | 374 | 7510 | 80.5 | 15.2 |
| Zm D2 | 364 | 20.0 | 18.2 | 993 | 18100 | 103 | 49.7 |
| Zm D2-15 | 472 | 21.8 | 21.7 | 1680 | 36400 | 102 | 77.1 |
| Zm D2-64 | 478 | 19.6 | 24.4 | 938 | 22900 | 101 | 47.9 |
| Zm D2-28 | 482 | 15.8 | 30.5 | 703 | 21500 | 120 | 44.5 |
| Zm D2-82 | 625 | 26.3 | 23.8 | 1300 | 30900 | 124 | 49.4 |
| Zm D2-67 | 568 | 20.0 | 28.4 | 955 | 27100 | 119 | 47.7 |
| Zm D2-68 | 554 | 22.1 | 25.1 | 1490 | 37400 | 113 | 67.5 |
| Zm D2- | 593 | 34.7 | 17.1 | 1330 | 22710 | 132 | 38.3 |
| 3P124 | | | | | | | |
| Zm F3 | 472 | 71.5 | 6.61 | 1100 | 7280 | 39.3 | 15.4 |
| Zm F3-88 | 693 | 55.6 | 12.6 | 2443 | 29600 | 85.7 | 44.0 |

In total, 303 neutral or beneficial mutations were identified at 136 positions. In only 16 instances was the mutation observed in more than one backbone sequence, inferring that the value of most of the mutations identified is specific for the amino acid sequence context in which it is placed.

Many more variants improved relative to C1 have been identified, but characterized only with regard to k_{cat} and kcat gly. These are shown in Table 3.

TABLE 3
Values for kcat and kcat gly for variants
improved relative to Zm C1.

| Variant | kcat | kcat gly | Mutations vs Zm D2 |
|---|---|---|---|
| 5P127 | 416 | 146 | 54V 241R 311L 382D |
| 2P083 | 408 | 143 | 54E 109V 113V 177Q 308A |
| 5P126 | 373 | 140 | 54D 303A |
| 2P097 | 487 | 138 | 54N 72Q 349M |
| 1P097 | 410 | 137 | 54N 72Q 349M |
| 5P041 | 471 | 137 | 54D 382D |
| 5P097 | 497 | 137 | 54D 87T 279V 308S |
| 5P039 | 386 | 136 | H69T |
| 5P112 | 365 | 136 | 248V 308A 419E |
| 4P009 | 371 | 136 | 54G 190S 416G |
| 3P118 | 259 | 135 | 72G |
| 5P110 | 422 | 133 | 38A 39G 54D 91G 164R 308A 419E |
| 1P113 | 410 | 133 | 246G 247Q |
| 5P136 | 413 | 133 | 54D 303A 316M |
| 4P065 | 392 | 132 | 54L 71P 72E 297S 416G |
| 2P001 | 404 | 131 | 54E 79C 241A |
| 5P004 | 402 | 131 | 54D 241A 297S |
| 5P005 | 430 | 129 | 54D |
| 5P102 | 434 | 129 | 54D 89F 310S 379D |
| 5P125 | 404 | 129 | 54D 74L 87T 127M 311S 379D |
| 5P104 | 462 | 127 | 54D 279V 310S |
| 5P100 | 381 | 127 | 54V 65V 69T 308S 394A |
| 5P113 | 396 | 125 | 54V 118C 164R 226G 248V 311S |
| 4P005 | 419 | 122 | 54E 72K 88G 224R 297S |
| 5P118 | 428 | 121 | 54D 92E 226G 246R 308S 316M 379D |
| 1P009 | 386 | 120 | 54N 279A 379D |
| 5P048 | 409 | 118 | 54V |
| 2P004 | 386 | 117 | 54E 62V 89F 109V |
| 5P027 | 422 | 117 | 54D 279V 297S |
| 5P002 | 391 | 116 | 54D 127M 279V 394A |
| 2P022 | 388 | 115 | 49D 79R 196E 279A |
| 5P018 | 397 | 115 | 54D 241A 437G |
| 4P058 | 381 | 113 | 54E 386V 416G |
| 5P053 | 382 | 113 | 54D 65V 118C 311S 437G |
| 4P123 | 383 | 112 | 54E 304C 416G |
| 5P010 | 427 | 110 | 54D 87T 91G 113A |
| 5P011 | 431 | 110 | 54D 89F 437G |
| 4P099 | 396 | 109 | 20E 71P 219G 247L 297S |

### Example 4

### Maize EPSPS Mutations and Mapping onto EPSPS from Other Crop Species

Mutations present in representative maize EPSPS variants (Zm) were identified in the context of the enzyme from rice (Os) (Oryza sativa; AF413082) by aligning the native enzymes and intended variants (Table 4).

TABLE 4
Design of variants in rice EPSPS containing mutations
identified through optimization of maize EPSPS.

| | | | | | | | | | | | | | | |
|---|---|---|---|---|---|---|---|---|---|---|---|---|---|---|
| Zm position | 2 | 3 | 4 | 13 | 18 | 20 | 35 | 54 | 58 | 61 | 62 | 69 | 72 | 84 |
| Os position | 2 | 3 | 4 | 13 | 18 | 20 | 35 | 54 | 58 | 61 | 62 | 69 | 72 | 84 |
| Zm native | A | G | A | K | T | K | A | H | G | R | T | A | A | K |
| Os native | A | K | A | R | A | Q | S | H | E | K | A | A | V | K |
| Zm C1 | | G | | K | T | K | A | H | G | R | T | A | A | |
| Os C1 | | K | | R | A | Q | S | H | E | K | A | A | V | |
| Zm D2 | | G | | K | T | K | A | H | G | R | T | | A | |
| Os D2 | | K | | R | A | Q | S | H | E | K | A | | V | |
| Zm D2-67 | | K | | K | T | K | A | H | G | R | T | | A | |
| Os D2-67 | | K | | R | A | Q | S | H | E | K | A | | V | |
| Zm F3 | A | G | A | K | T | K | A | H | G | R | T | A | A | K |
| Os F3 | A | K | A | R | A | Q | S | H | E | K | A | A | V | K |
| Zm F3-88 | A | G | A | K | T | K | A | | G | R | T | A | A | K |
| Os F3-88 | A | K | A | R | A | Q | S | | E | K | A | A | V | K |

| | | | | | | | | | | | | | | |
|---|---|---|---|---|---|---|---|---|---|---|---|---|---|---|
| Zm position | | 92 | 98 | 102 | 155 | 162 | 208 | 216 | 226 | 243 | 246 | 274 | 297 | 302 |
| Os position | 89 | 93 | 99 | 103 | 156 | 163 | 209 | 217 | 227 | 244 | 247 | 275 | 298 | 303 |
| Zm native | — | E | L | G | D | N | I | R | E | K | K | E | K | E |
| Os native | K | E | L | G | E | K | I | R | E | K | G | Q | K | D |
| Zm C1 | — | E | | | D | N | | R | E | | K | E | K | E |
| Os C1 | K | E | | | E | K | | R | E | | G | Q | K | D |
| Zm D2 | | | | | D | N | | R | E | | K | E | K | E |
| Os D2 | K | | | | E | K | | R | E | | G | Q | K | D |
| Zm D2-67 | — | | | | D | N | | R | E | | K | E | K | E |
| Os D2-67 | K | | | | E | K | | R | E | | G | Q | K | D |
| Zm F3 | — | E | | | D | N | | R | E | K | K | E | K | E |
| Os F3 | K | E | | | E | K | | R | E | K | G | Q | K | D |
| Zm F3-88 | — | E | | | D | N | | | | K | K | E | | E |
| Os F3-88 | K | E | | | E | K | | | | K | G | Q | | D |

| | | | | | | | | | | | | | |
|---|---|---|---|---|---|---|---|---|---|---|---|---|---|
| Zm position | 315 | 317 | 323 | 333 | 354 | 361 | 391 | 395 | 402 | 417 | 429 | 434 | 444 |
| Os position | 316 | 318 | 324 | 334 | 355 | 362 | 392 | 396 | 403 | 418 | 430 | 435 | 445 |
| Zm native | F | R | I | V | A | T | E | V | D | E | R | D | K |
| Os native | Y | K | V | V | A | T | E | I | D | D | R | N | R |
| Zm C1 | F | R | I | V | A | T | | V | | E | R | D | K |
| Os C1 | Y | K | V | V | A | T | | I | | D | R | N | R |
| Zm D2 | F | R | I | | A | T | | V | | E | R | D | K |
| Os D2 | Y | K | V | | A | T | | I | | D | R | N | R |
| Zm D2-67 | F | R | I | | | T | | V | | E | R | D | K |
| Os D2-67 | Y | K | V | | | T | | I | | D | R | N | R |
| Zm 13 | F | R | I | V | A | | E | V | | E | R | D | K |
| Os F3 | Y | K | V | V | A | | E | I | | D | R | N | R |
| Zm F3-88 | F | R | I | | A | | E | V | | E | | D | K |
| Os F3-88 | Y | K | V | | A | | E | I | | D | | N | R |

Only variable positions are shown. Naturally occurring differences in the rice sequence from the maize sequence are shown. The mutations designed to create rice orthologs of selected maize variants are shown.
Genes designed to encode the rice orthologs were synthesized by a commercial provider and expressed and purified as described above. Fitness was assessed by determining the values for kcat gly and shown in Table 5.

TABLE 5
Fitness of rice EPSPS variants in the presence of glyphosate.

| Variant | kcat gly | Zm/Os |
|---|---|---|
| Zm C1 | 100 | 1.50 |
| Os C1 | 66.5 | |
| Zm D2 | 114 | 1.28 |
| Os D2 | 89.0 | |
| Zm D2-67 | 138 | 1.35 |
| Os D2-67 | 102 | |
| Zm F3 | 45.2 | 1.16 |
| Os F3 | 39.0 | |
| Zm F3-88 | 98 | 1.57 |
| Os F3-88 | 62.3 | |

Fitness is indicated by the parameter kcat gly, defined in Example 1.
This table 5 demonstrates that the mutations identified in the process of optimizing maize EPSPS have a substantially similar effect in the context of the rice enzyme.

The mutations discovered in the course of optimizing maize EPSPS were used to optimize the soybean EPSPS enzyme. Soybean has two genes coding for EPSPS, one found on chromosome 1 (GenBank #NC_016088.2) and the other on chromosome 3 (NC_016090.2). The mutations in maize H6 and C1 were mapped onto the enzyme coded by the NC_016088.2 gene and aligned with the native Chrom1 and Chrom3 enzymes. Oligonucleotides were designed so as to allow any of the amino acids available at the variable positions (bold, Table 6) to combine randomly.

TABLE 6
Design of soybean EPSPS combinatorial library based
on mutations present on maize variants H6 and C1.

| | | | | | | | | |
|---|---|---|---|---|---|---|---|---|
| Zm position | 2 | 4 | 72 | 84 | 98 | 102 | 208 | 243 |
| Zm wt | A | A | A | K | L | G | I | K |
| Zm C1 | R | W | A | R | C | A | L | E |
| Zm H6 | R | W | Q | R | C | A | L | K |
| Gm chrom1 | S | S | T | L | L | G | V | K |
| Gm chrom3 | S | A | T | L | L | G | V | K |
| Library | RS | AWS | AQT | RL | CL | A | ILV | EK |
| design | | | | | | | | |
| Gm1 position | 5 | 7 | 78 | 90 | 105 | 109 | 215 | 250 |

| | | | | | | | | |
|---|---|---|---|---|---|---|---|---|
| Zm position | 279 | 302 | 361 | 391 | 402 | 416 | 438 | 441 |
| Zm wt | T | E | T | E | D | A | V | T |
| 123-C1 | T | E | T | P | G | A | V | T |
| 868-H6 | A | S | S | G | G | G | R | Q |
| Gm chrom1 | S | E | T | E | D | G | V | R |
| Gm chrom3 | N | E | T | E | D | G | V | R |
| Library | TASN | ES | TS | PGE | GD | AG | RV | TQR |
| design | | | | | | | | |
| Gm1 position | 286 | 309 | 368 | 398 | 409 | 423 | 445 | 448 |

The library was screened as described above. A variant designated F3 had a value for kcat gly of 56.3 min⁻¹. Saturation mutagenesis was performed as described above and the following mutations were identified as being neutral or beneficial by the criterion of having a value for kcat gly that is 80% of that of Gm F3.

TABLE 7
Neutral or beneficial diversity identified by
performing saturation on Gm EPSPS variant F3.

| | Native | | Neutral, |
|---|---|---|---|
| | amino | F3 | beneficial |
| Position | acid | backbone | diversity |
| 10 | S | | Q |
| 96 | E | | W |
| 105 | L | C | |
| 109 | G | A | |
| 152 | G | | E |
| 158 | F | | M |
| 174 | L | | V |
| 181 | S | | C |
| 184 | S | | I |
| 212 | L | | V |
| 215 | V | | L |
| 229 | G | | L |
| 237 | N | | G |
| 238 | W | | R |
| 250 | K | | R E |
| 254 | N | | R |
| 256 | F | | H |
| 285 | T | | V |
| 293 | K | | R |
| 299 | E | | A |
| 300 | K | | I |
| 309 | E | | S |
| 320 | D | | V |
| 336 | K | | A |
| 340 | V | | A |
| 352 | N | | F R |
| 368 | T | S | T |
| 379 | R | | Q |
| 396 | P | | G |
| 397 | P | | G |
| 402 | V | | D |
| 409 | D | G | E |
| 422 | C | | A |
| 423 | G | | E |
| 426 | P | | V |
| 436 | R | | G |

A library was constructed and screened as above. Because the change of V to A at position 340 conferred a significant fitness improvement over Gm F3 (kcat gly=56.3), it was used as the backbone for the combinatorial library. The library was constructed, screened and evaluated by the methods described in Examples 1 and 2. One variant (Gm F3-02-A7) was significantly improved, having a value for kcat gly of 80.3 min-1.

### Example 5

### Maize EPSPS Mutations of Variants Designated as Zm D2, Zm D2-64, Zm D2-67, Zm D2-3P124, Zm D2-68, Zm F3 and Zm F3-88 are Transferable to EPSPS from Other Plant Species

An alignment of the amino acid sequences of EPSPS from various plant species shows a level of homology ranging from 80% to 99%, suggesting that the mutations defined in the maize background would have a similar effect in EPSPS from other species. The alignments shown herein are used to map the Zm D2, Zm D2-64, Zm D2-67, Zm D2-3P124, Zm D2-68, Zm F3 and Zm F3-88 mutations onto the EPSPS sequences from rice, wheat, soybean, sorghum, brassica, tomato, potato, cotton, millet, barley, and other commercially important crop species.

Native EPSPS amino acid sequences of rice (Oryza sativa) (SEQ ID NO: 22), sorghum (Sorghum halepense) (SEQ ID NO: 23), and sunflower (Helianthus annus) (SEQ ID NO: 24 or 36) including the chloroplast transit peptide sequences were assembled and analyzed for mapping the correponding amino acid mutations from the maize EPSPS variants disclosed herein.

### Example 6

### Production of Glyphosate-Resistant Maize Expressing Glyphosate Tolerant Plant EPSPS

Maize plants expressing EPSPS variant genes are produced using at least two approches—(i) recombinant DNA-based transformation or site-directed changes at the endogenous EPSPS genomic locus. Recombinant DNA based transformation methods are well known in the art, e.g. Agrobacterium tumefaciens-mediated and particle bombardment based transformations.

(i) Recombinant Maize EPSPS-Variant Transformation

Agrobacterium tumefaciens based plant transformation vectors are constructed according to methods known in the art. EPSPS vectors contain a T-DNA insert having a constitutive plant promoter, such as an ubiquitin promoter, an intron, an optional enhancer such as a 35S enhancer element or other plant derived enhancer elements, an EPSPS variant DNA encoding a glyphosate tolerant EPSPS (e.g., Zm D2, Zm D2-64, Zm D2-67, Zm D2-3P124, Zm D2-68, Zm F3 and Zm F3-88), and a plant terminator such as, for example, a PinII terminator. Maize immature embryos are excised and infected with an Agrobacterium tumefaciens vector containing the EPSPS variant of interest. After infection, embryos are transferred and cultured in co-cultivation medium. After co-cultivation, the infected immature embryos are transferred onto media containing 1.0 mM glyphosate. This selection generally lasts until actively growing putative transgenic calli are identified. The putative transgenic callus tissues are sampled using PCR and optionally a Western assay to confirm the presence of the EPSPS variant gene. The putative transgenic callus tissues are maintained on 1.0 mM glyphosate selection media for further growth and selection before plant regeneration. At regeneration, callus tissue confirmed to be transgenic are transferred onto maturation medium containing 0.1 mM glyphosate and cultured for somatic embryo maturation. Mature embryos are then transferred onto regeneration medium containing 0.1 mM glyphosate for shoot and root formation. After shoots and roots emerge, individual plantlets are transferred into tubes with rooting medium containing 0.1 mM glyphosate. Plantlets with established shoots and roots are transplanted into pots in the greenhouse for further growth, to obtain TO spray data, and to produce T1 seed.

In order to evaluate the level of glyphosate resistance of the transgenic maize plants expressing the EPSPS variant transgenes, TO plants are sprayed with glyphosate in the greenhouse. Glyphosate concentrations include dosage of e.g., 1× rate of a commercially available glyphosate formulation. Plant resistance levels are evaluated by plant discoloration scores and plant height measurements. Plant discoloration is evaluated according to the following scale:

Discoloration Score at 1, 2, 3 and 4 Weeks After Spray with Glyphosate
9=no leaf/stem discoloration
7=minor leaf/stem discoloration
5=worse leaf/stem discoloration
3=severely discolored plant or dying plant
1=dead plant

Plant Height Measurements are recorded before spraying with glyphosate and after spraying with glyphosate at 1, 2, 3 and 4 weeks post-application. Two plants are sent to the greenhouse from each event (independent transgenic callus). Plant 1 is kept for seed production and is not sprayed with glyphosate. Plant 2 is sprayed at 2×-4× glyphosate (1× glyphosate=26 ounces/acre) at 14 days after transplanting. The TO plant discoloration scores at 7 and 14 days after the spray are also observed. Height data at tasseling is also measured.

(ii) Guided Cas9-Based EPSPS Modifications

Expression cassettes for guide RNA/Cas endonuclease based genome modification in maize plants are disclosed at least in Examples 1-15 of International Application No. PCT/US2015/38767, filed Jul. 1, 2015 and herein incorporated by reference.

Described herein is a guide RNA/Cas endonuclease system that is based on the type II CRISPR/Cas system and includes a Cas endonuclease and a guide RNA (or duplexed crRNA and tracrRNA) that together can form a complex that recognizes a genomic target site in a plant and introduces a double-strand-break into said target site (U.S. patent application 61/868,706, filed Aug. 22, 2013), incorporated herein by reference. In this Example, the desired target site is the maize endogenous native EPSPS genomic sequence.

The maize optimized Cas9 endonuclease and single guide RNA expression cassettes containing the specific maize variable targeting domains are co-delivered to e.g., 60-90 Hi-II immature maize embryos by particle-mediated delivery using techniques well known in the art and optionally, in the presence of BBM and WUS2 genes (U.S. patent application Ser. No. 13/800,447, filed Mar. 13, 2013).

After 7 days, the 20-30 most uniformly transformed embryos are pooled and total genomic DNA is extracted. The region surrounding the intended target site is PCR amplified with Phusion® High Fidelity PCR Master Mix (New England Biolabs, M0531L) adding on the sequences necessary for amplicon-specific barcodes and Illumnia sequencing using “tailed” primers through two rounds of PCR.

The resulting PCR amplifications are purified with a Qiagen PCR purification spin column; the concentration is measured with a Hoechst dye-based fluorometric assay; the PCR amplifications are combined in an equimolar ratio; and single read 100 nucleotide-length deep sequencing is performed using Illumina's MiSeq Personal Sequencer with a 30-40% (v/v) spike of PhiX control v3 (Illumina, FC-110-3001) to off-set sequence bias. Only those reads with a ≥1 nucleotide indel arising within the 10 nucleotide window centered over the expected site of cleavage and not found in a similar level in the negative control are classified as non homologous end-joining mutations. NHEJ mutant reads with the same mutation are counted and collapsed into a single read and the top 10 most prevalent mutations are visually confirmed as arising within the expected site of cleavage. The total numbers of visually confirmed NHEJ mutations are then used to calculate the % mutant reads based on the total number of reads of an appropriate length containing a perfect match to the barcode and forward primer.

The frequency of NHEJ mutations recovered by deep sequencing for the guide RNA/Cas endonuclease system targeting the one or more desired EPSPS targets (e.g., one or more mutations of the Zm D2, Zm D2-64, Zm D2-67, Zm D2-3P124, Zm D2-68, Zm F3 and Zm F3-88 variants) compared to the cas9 only control is analyzed. This Example describes that the guide RNA/Cas9 endonuclease system described herein can be used to introduce a double strand break at genomic sites of interest within the maize endogenous EPSPS genomic regions. Editing the EPSPS target results in the production of plants that are tolerant and/or resistant against glyphosate based herbicides.

### Example 7

### Efficacy of Shuffled Plant EPSPS for Conferring Glyphosate Tolerance in Transformed Plants

Transformation vectors are constructed that include nucleotide sequences coding for either the native maize EPSPS or maize EPSPS variants including Zm D2, Zm D2-64, Zm D2-67, Zm D2-3P124, Zm D2-68, Zm F3 and Zm F3-88. Each is preceded by nucleotide sequences coding for either an Arabidopsis chloroplast targeting peptide or an artificial CTP termed 6H1 (U.S. Pat. No. 7,345,143). The resulting four CTP-enzyme combinations are preceded either by the native Arabidopsis EPSPS promoter (AT1G48860), the ubiquitin-3 promoter, or the ubiquitin-10 promoter (Norris et al. 1993. Plant Mol Biol 21:895-906) for multiple combinations of promoter, CTP and enzyme.

Transformation vectors containing constitutive promoters for expression in maize, wheat, rice, sorghum, sunflower, cotton, soybean, barley, millet, cereals are constructed and suitable transformation procedures are used to obtain plant cells stably transformed with polynucleotides that confer glyphosate tolerance.

### Example 8

### Efficacy of Shuffled Plant EPSPS for Conferring Glyphosate Tolerance in Transformed Soybean

Transformation vectors are constructed that included nucleotide sequences coding for either the native soybean EPSPS or the variant soybean EPSPS sequences provided as SEQ ID NO:13, SEQ ID NO:14, SEQ ID NO:15, respectively). Each is preceded by nucleotide sequences coding for an artificial CTP termed 6H1. The resulting CTP-enzyme combinations are preceded appropriate plant operable promoters. The glyphosate tolerant mutations in the soy EPSPS sequences are shown below in Table 8:

TABLE 8
Corresponding positions of EPSPS mutations in soy.

| | Amino acid positions | | | | | | |
|---|---|---|---|---|---|---|---|
| Variant | 99 | 105 | 109 | 300 | 340 | 368 | 409 |
| Gm native EPSPS, | D | L | G | K | V | T | D |
| chrom1 | | | | | | | |
| Gm F3 | D | C | A | K | V | S | G |
| Gm F3-V340A | D | C | A | K | A | S | G |
| Gm F3-02A7 | G | C | A | I | A | S | G |

Binary vectors for Agrobacterium mediated transformation are constructed using standard molecular biology techniques. Glycine max (93Y21) hairy root transformation is carried out using a method slightly modified from that of Cho et al. (Cho et al. 2000. Planta 210:195-204), in which the wounded cotyledon explants are infected with a suspension of Agrobacterium rhizogenes strain K₅₉₉ transformed with binary vectors described.

### Example 9

### Endogenous Genome Editing of EPSPS Gene Locus

Maize optimized Cas9 endonucleases are developed and evaluated for their ability to introduce one or more double-strand breaks at the EPSPS genomic target sequence that correspond to the variants designated Zm D2, Zm D2-64, Zm D2-67, Zm D2-3P124, Zm D2-68, Zm F3 and Zm F3-88. A maize optimized Cas9 endonuclease (moCas9) is generally supplemented with a nuclear localization signal (e.g., SV40) by adding the signal to the 5′ end of the moCas9 coding sequence. The plant moCas9 expression cassette is subsequently modified by insertion of an intron into the moCas9 coding sequence in order to enhance its expression in maize cells and to eliminate its expression in E. coli and Agrobacterium. The maize ubiquitin promoter and the potato proteinase inhibitor II gene terminator sequences complement the moCas9 endonuclease gene designs. However, any other promoter and/or terminator can be used.

A single guide RNA (sgRNA) expression cassette includes for example, U6 polymerase Ill maize promoter and its cognate U6 polymerase Ill termination sequences. The guide RNA includes a nucleotide variable targeting domain followed by a RNA sequence capable of interacting with the double strand break-inducing endonuclease.

A maize optimized Cas9 endonuclease target sequence (moCas9 target sequence) within the EPSPS codon sequence is complementary to the nucleotide variable sequence of the guide sgRNA, which determines the site of the Cas9 endonuclease cleavage within the EPSPS coding sequence. This targeting region can vary based on the nature and the number of mutations to be targeted within the EPSPS locus.

The moCAS9 target sequence is synthesized and cloned into the guide RNA-Cas9 expression vector designed for delivery of the components of the guide RNA-Cas9 system to the maize cells through Agrobacterium-mediated transformation. Agrobacterium T-DNA also delivers the yeast FLP site-specific recombinase and the WDV (wheat dwarf virus) replication-associated protein (replicase), if needed. If the moCas9 target sequences are flanked by the FLP recombination targets (FRT), they can be excised by FLP in maize cells forming episomal (chromosome-like) structures. Such circular DNA fragments are replicated by the WDV replicase (the origin of replication was embedded into the WDV promoter) allowing their recovery in E. coli cells. If the maize optimized Cas9 endonuclease makes a double-strand break at the moCas9 target sequence, its repair might produce mutations. The procedure is described in detail in: Lyznik, L. A., Djukanovic, V., Yang, M. and Jones, S. (2012) Double-strand break-induced targeted mutagenesis in plants. In: Transgenic plants: Methods and Protocols (Dunwell, J. M. and Wetten, A. C. eds). New York Heidelberg Dordrecht London: Springer, pp. 399-416. The maize optimized Cas9 endonuclease described herein is functional in maize cells and efficiently generates double-strand breaks at the moCas9 target sequence.

In order to accomplish targeted genome editing of the maize chromosomal EPSPS gene, a polynucleotide modification template for editing the EPSPS coding sequence may be created and co-delivered with the guide RNA/Cas9 system components. There can be more than one modification template delivered simultaneously or sequentially.

A polynucleotide modification template includes one or more nucleotide modifications (e.g., nucleotide changes that correspond to the one or more amino acid changes disclosed herein) when compared to the native EPSPS genomic sequence to be edited. These nucleotide modifications are generally substitution mutations. The EPSPS template sequences may encode a functional EPSPS protein or may be partial fragments that do not encode a full-length functional polypeptide.

The EPSPS polynucleotide modification template may be co-delivered with the guide sgRNA expression cassette and a maize optimized Cas9 endonuclease expression vector, which contains the maize optimized Cas9 endonuclease expression cassette and a selectable marker gene, using particle bombardment. Ten to eleven day-old immature embryos are placed embryo-axis down onto plates containing N6 medium and are incubated at 28° C. for 4-6 hours before bombardment. The plates are placed on the third shelf from the bottom in the PDS-1000 apparatus and bombarded at 200 psi. Post-bombardment, embryos are incubated in the dark overnight at 28° C., transferred to plates containing N6-2 media, and then stored for 6-8 days at 28° C. The embryos are then transferred to plates containing N6-3 media for three weeks. Responding callus is then transferred to plates containing N6-4 media for an additional three-week selection. After six total weeks of selection at 28° C., a small amount of selected tissue is transferred onto the MS regeneration medium and incubated for three weeks in the dark at 28° C.

Multiple callus events selected on media containing appropriate substrate for the selectable marker (e.g., bialophos for the moPAT selectable marker gene) are screened for the presence of the targeted point mutations. Further sequencing of the EPSPS locus is performed to confirm the mutations. Plantlets are generated from the callus events following standard procedures.

### Example 10

### Comparative Kinetics of Various Maize EPSPS

Combinatorial shuffling, maize native EPSPS—The mutations discovered were used to construct a combinatorial library designed to explore combinations of the desensitizing mutations in novel sequence contexts provided by the newly identified neutral mutations. Note that the amino acid position numbering in this Example 10 refers to the relative position of native maize EPSPS without the N-terminal Met. Therefore, G101A in this Example would be G102A in reference to SEQ ID NO: 1, wherein the maize EPSPS sequence has a Met added at the N-terminal. The diversity used was the same with the addition of G101A, T102 (IALGV) and P106(WA). In all, 43 substitutions at 29 positions were selected. The library was synthesized entirely from oligonucleotides, using the known technique of synthetic shuffling and was termed NatFS (Native, fully synthetic). The vector DNA of the library was transformed into the BL21(DE3) Tuner-AroA knockout strain and the cells were plated onto M9 medium containing either 30 mM glyphosate and 30 μM of the lac operon inducer isopropyl β-D-1-thiogalactopyranoside (IPTG) or 50 mM glyphosate and no IPTG. The 184 colonies that grew on either plate were picked and subjected to a second tier of screening in which EPSPS proteins were purified and activity measured at high (200 μM) and low (50 μM) PEP and S3P, with or without 10 μM glyphosate. Selected variants were subjected to substrate saturation kinetic analysis.

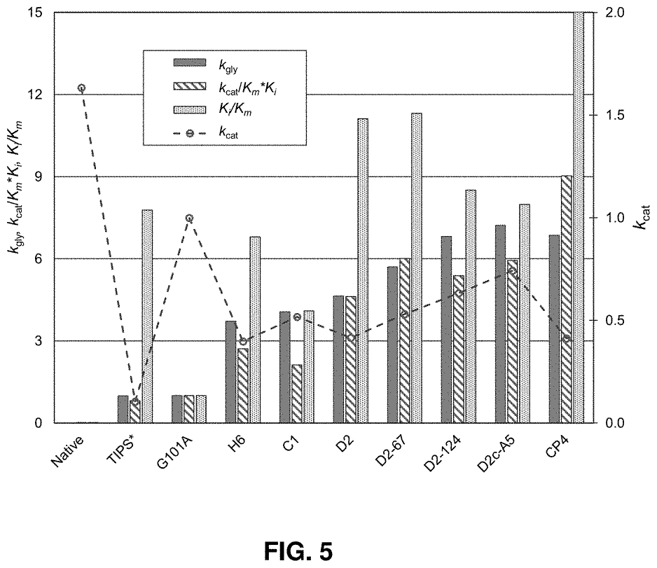

A parameter, k_{gly}, was devised because it takes into account anticipated concentrations of substrates and inhibitor, would better capture enzyme fitness under the conditions of the application than k_{cat}/Kₘ*Kᵢ. FIG. 5 shows that fitness as judged by those two options correlate rather well with a few exceptions. CP4, with its very high Kᵢ, displayed a disproportionately high k_{cat}/Kₘ*Kᵢ. This is due to the greater impact of Kᵢ on k_{cat}/Kₘ*Kᵢ compared with its impact on the velocity equation for competitive inhibition, ν=kcat[E][S]/Km(1+[I]/Kᵢ)+[S], which k_{gly} parameter seeks to represent. The proportionately lower value with CP4 for k_{gly} is due to its low k_{cat}. The most common outliers from the k_{gly} trendline are with variants with exceptional selectivity, especially TIPS and CP4. With those variants, the underperformance is a function of a deficient k_{cat}. The converse, good k_{cat} but poor selectivity (G101A), is likewise unsatisfactory. Variant D2c-A5 incorporates the combination of the parameters under optimization.

A value of 66 nM for Kᵢ for the native enzyme is in accord with the 80 nM reported for EPSPS from Pisum sativum and the 48 nM obtained with the Eleusine indica enzyme. These values are generally lower than the low μM values seen with bacterial enzymes. Though glyphosate has not been considered a “slow-tight binding” inhibitor, its release from a E:S3P:glyph complex was slow enough to be observed over a 40-sec span.

P106x and TIPS variants were not improved—Variants NatFS-B, -D and -E each include one of the previously known mutations or pair of mutations that reduce sensitivity of EPSPS to inhibition by glyphosate. Along with three other mutations, NatFS-D has leucine substituted for proline at position 106. Alone, the P106L mutation raised Kᵢ for glyphosate 60-fold, but also raised Kₘ for PEP 5-fold (Table 9). The three additional mutations present in NaFS-D served to lower Kₘ for PEP from 47 μM, seen with P106L alone, to 10.3 μM with 60% retention of Kᵢ. The overall result was 30-fold improved fitness (k_{cat}/Kₘ*Kᵢ) compared to native maize EPSPS.

Along with two other mutations, NatFS-B contains the T102I and P106S (TIPS) mutations present in the GA21 maize transformation event. Kinetic analysis indicated that the T102I and P106S mutations in variant NatFS-B confer a high level of insensitivity to glyphosate while retaining near native affinity for PEP but with only ˜5% of the native k_{cat} (Table 9), confirming results obtained previously. NatFS-D and -B were each subjected to a cycle of saturation mutagenesis and combinatorial shuffling, attempting to improve the Kᵢ of NatFS-D or to improve the k_{cat} of NatFS-B. Neither attempt was successful. Under the experimental conditions tested, mutation(s) that could work in concert with P106L (and presumably P106S or A) to enable any further desensitization, were not identified. Further, mutation(s) that could compensate for the low k_{cat} imposed by the TIPS mutations, were also not identified under the experimental conditions tested.

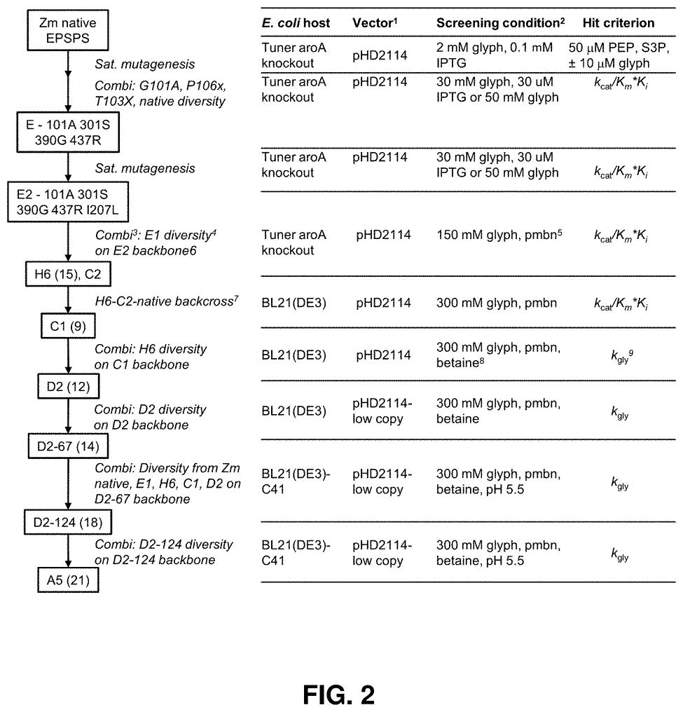

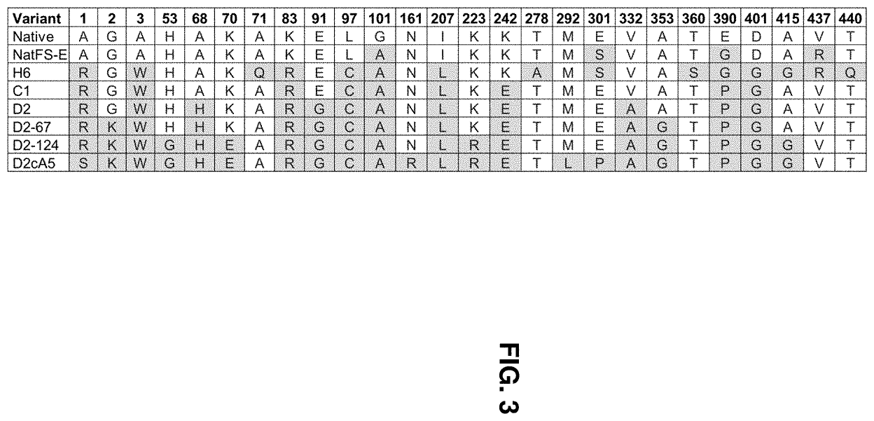

Further optimization of maize EPSPS-G102A —In the context of the maize enzyme, G101A is highly insensitive to glyphosate, but has 35-fold elevated Kₘ for PEP relative to native EPSPS (Table 9), confirming earlier results with Class I EPSPS. However, unlike the situation with variants containing P106L or the TIPS mutations, G101A was amenable to improvement through iterative cycles of diversity generation and combinatorial shuffling. The process is shown schematically in FIG. 2. Progressive addition of mutations to G101A were obtained. The kinetic parameters for NatFS-E are poorer than those of G101A (Table 9), suggesting that one or more of its three additional mutations (E301S, E390G, V437R) was detrimental. Interestingly, all three were eliminated in the H6-C2-native backcross (see Supporting Information for details). Five of the six substitutions eliminated from H6 by the H6-C2-native backcross procedure (including the three from NatFS-E) did not reappear in subsequent cycles of saturation mutagenesis and combinatorial shuffling (FIG. 3). Evaluated mutations other than G101A are outside the active site. An alignment of the complete sequences provides a view of the areas in which the substitutions occur in relation to the positions where amino acids have been identified or strongly implicated as having roles in substrate binding or catalysis. The progressive addition of mutations to G101A had the effect of restoring affinities (Kₘ) of PEP and S3P nearly to those of native EPSPS while maintaining much of the insensitivity (Kᵢ) to glyphosate (Table 9).

TABLE 9
Kinetic parameters of variants with known desensitizing
mutations and key variants in maize EPSPS optimization

| | k_{cat}, | Kₘ | | | Kₘ | k_{gly}¹, | |
|---|---|---|---|---|---|---|---|
| | min⁻¹ | PEP, μM | k_{cat}/Kₘ | Kᵢ, μM | S3P, μM | min⁻¹ | k_{cat}/Kₘ*Kᵢ² |
| Zm native | 1630 ± 14 | 9.5 ± 0.3 | 172 | 0.066 ± 0.003 | 13.2 ± 0.6 | nd | 11 |
| P106S | 1540 ± 12 | 11.5 ± 0.5 | 134 | 0.33 ± 0.02 | 15.4 ± 0.4 | 2.3 ± 0.07 | 44 |
| P106L | 1760 ± 10 | 47.0 ± 1.1 | 37.5 | 3.94 ± 0.17 | 27.6 ± 0.7 | 5.7 ± 0.17 | 148 |
| NatFS-D³ | 1450 ± 22 | 10.3 ± 0.7 | 140 | 2.34 ± 0.18 | 17.5 ± 0.6 | 10.1 ± 0.1 | 329 |
| NatFS-B⁴ | 105 ± 1.0 | 16.2 ± 0.7 | 6.5 | 731 ± 38 | 27.5 ± 0.7 | 25.5 ± 0.4 | 4740 |
| G101A | 1000 ± 35 | 333 ± 26 | 3.0 | 1930 ± 40 | 84.0 ± 2.5 | 25.7 ± 0.2 | 5780 |
| NatFS-E⁵ | 824 ± 15 | 347 ± 6.8 | 2.4 | 1430 ± 137 | 153 ± 3.9 | 17.7 ± 0.6 | 3400 |
| H6 | 397 ± 4 | 25.1 ± 0.9 | 15.8 | 989 ± 31 | 14.0 ± 0.5 | 95.5 ± 2.0 | 15700 |
| C1 | 517 ± 7 | 18.9 ± 0.5 | 27.4 | 449 ± 48 | 11.4 ± 0.5 | 104 ± 1.6 | 12300 |
| D2 | 414 ± 38 | 14.5 ± 2.2 | 28.6 | 935 ± 66 | 11.3 ± 0.3 | 119 ± 2.0 | 26700 |
| D2-67 | 530 ± 17 | 14.4 ± 0.6 | 36.8 | 945 ± 98 | 10.9 ± 0.4 | 146 ± 2.0 | 34800 |
| D2-124 | 631 ± 5 | 18.1 ± 0.7 | 34.9 | 893 ± 53 | 11.1 ± 0.7 | 175 ± 2.5 | 31130 |
| D2c-A5 | 741 ± 6 | 18.1 ± 0.6 | 40.9 | 839 ± 40 | 12.6 ± 1.4 | 186 ± 4.7 | 34350 |
| CP4⁶ | 411 ± 5 | 15.5 ± 0.4 | 26.5 | 1970 ± 276 | 5.2 ± 0.5 | 176 ± 1.6 | 52240 |

For assay procedure and analysis, see Experimental Procedures. Values for Kₘ were obtained by varying one substrate, with the other present at 10 times its Kₘ. The values shown are therefore regarded as apparent Kₘ.
¹Enzyme turnover (min⁻¹) at 30 μM PEP and S3P, 1 mM glyphosate (see Results for rational as a fitness parameter)
²Calculated with Kₘ for PEP
³Variant captured in library NatFS (see Results) having P106L plus three other mutations
⁴Variant captured in library NatFS having T102I, P106S and two other mutations
⁵Variant captured in library NatFS having G101A plus three other mutations
⁶EPSPS from Agrobacterium sp. strain CP4

A customized parameter for predicting performance in the treated plant that would be a more accurate representation than k_{cat}/Kₘ*Kᵢ. Viewed as dimensions for a volumetric measurement, k_{cat}, Ki and 1/Kₘ are useful for an initial evaluation of the capacity for catalysis in the presence of an inhibitor. However, k_{cat}/Kₘ*Kᵢ may be inadequate for predicting the reaction velocity under the conditions of the application (plants sprayed with glyphosate) because it may neglect concentrations of substrate and inhibitor, factors that are not intrinsic to the enzyme, but on which the reaction rate depends. Therefore, libraries derived from C1 on in FIG. 2 were evaluated with a single rate measurement designed to take all factors in the rate equation for competitive inhibition into account. The concentrations of PEP and S3P were set as nearly as possible (30 μM, limited by the sensitivity of our assay) to the presumed intracellular concentrations of 10-15 μM, the approximate values of Kₘ for both PEP and S3P for the native enzyme (Table 9). Glyphosate was included at 1 mM, a concentration attainable in tissues, especially meristems, receiving metabolite flow from treated leaves. The pH (7.0), ionic strength (100 mM KCl) and co-solvent concentration (5% ethylene glycol) were also intended to mimic in vivo conditions. The unit for the parameter is reaction rate (μM·min⁻¹) per enzyme concentration (μM), or min⁻¹, describing the enzyme turnover under application conditions, which we abbreviate as “k_{gly}”. Although individual kinetic parameters for key variants were obtained, k_{gly} was adopted a parameter needed both for medium throughput screening and for ultimate evaluation of fitness.

G101A was associated with a 30,00-fold increase in Ki, but also with a 35-fold increase in Km for PEP (Table 9). Alanine is present naturally at the homologous position in the Class II EPSPS from Agrobacterium sp. strain CP4. CP4 EPSPS exhibited a high degree of insensitivity to glyphosate but with a Km for PEP of just 15.5 μM (Table 9). Comparison of the crystal structures of CP4 ligated with S3P and glyphosate [PDB 2GGA] and E. coli EPSPS with the contextually equivalent glycine mutated to alanine ligated with S3P and glyphosate indicate that the alanine methyl group in CP4 is positioned 0.3 Angstroms further away from the phosphonate group of glyphosate than in the E. coli structure. Because PEP is shorter than glyphosate, it is hypothesized that the alanine methyl group in CP4 EPSPS is ideally positioned to interfere with binding of glyphosate but not PEP. Though there is only 24-26% homology between the CP4 enzyme and E. coli or maize EPSPS, structures of the CP4 and E. coli enzymes show that they share the same structural fold and topology. Presumably, the amino acid sequence of CP4 creates an overall structural context that places the alanine methyl group in its favorable position. Likewise, the highly improved parameters of variants D2-124 (shown in Table 10) and D2c-A5 relative to G101A alone presumably are the result of the mutations outside the active site acting to re-position the A101 methyl group by ˜0.3 Angstroms, for optimal discrimination between glyphosate and PEP. A comparison of crystal structures of maize native and variant EPSPS would provide an interesting verification.

TABLE 10
D2c variants with substitutions relative to D2-124.

| | kcat | Std. | |
|---|---|---|---|
| Variant | gly | Dev | Substitutions relative to D2-124 |
| D2c-106 | 159 | 4.1 | R2S S66T E88G N118R L208R M293L E302P |
| D2c-116 | 158 | 5.8 | R2F S66T E88G N162R K241R T279E M293L |
| | | | E302P |
| D2c-118 | 168 | 8.3 | R2S E88G A225V P311W |
| D2c-158 | 165 | 11.5 | R2S E88G N118R A225V K241R T308E |
| | | | P311W |
| D2c-170 | 162 | 3.0 | R2S N118R A225V K241R M293L E302S |
| D2c-171 | 164 | 6.1 | E88G N118R A225V P111V |
| D2c-173 | 170 | 3.3 | R2S S66T E88G A225V M293L T308A |
| D2c-200 | 159 | 7.9 | R2S T308E |
| D2c-230 | 163 | 6.8 | R2S E88G D136P M293L P311R |
| D2c-238 | 160 | 2.3 | R2S S66T V225V K241R T308E |
| D2c-152 | 156 | 7.3 | R2S N118R A225V E302A P311R |
| D2c-164a | 167 | 4.2 | S66T E88G K241R M293L E302P |
| D2c-171 | 159 | 4.3 | R2S S66T M293L P311R |
| D2c-178 | 163 | 7.0 | R2S E88G D136P M293L P311R |
| D2c-A5 | 156 | 5.4 | R2S N162R M293L E302P |

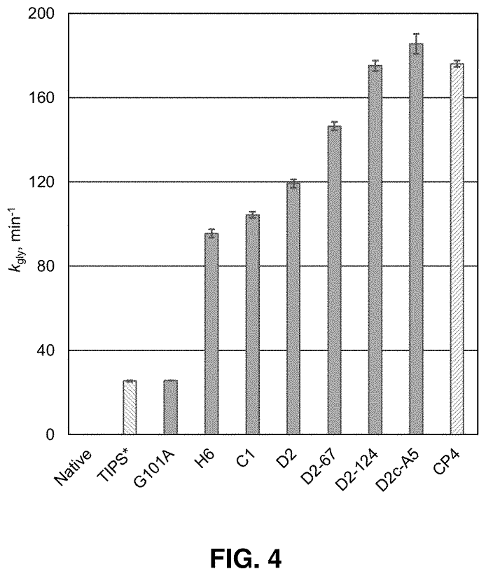

The progressive increase in k_{gly} is shown graphically in FIG. 4. The largest step in the progression was the 4-fold increase found in the first combinatorial library involving G101A. Variants from H6 on show reduced Kₘ for PEP, with some variants falling within 1.5-fold of the native value (Table 9). Most of the insensitivity to glyphosate conferred by the G101A mutation was retained, with Kᵢ values clustering around 900 μM for all variants but C1. Optimization culminated with variants D2-124 and D2c-A5, with 18 and 21 mutations, respectively.

### Example 11

### Mapping of Maize D2-124 Variant Mutations to Other Crop Species

An alignment of the amino acid sequences of EPSPS from 11 plant species shows identity at 66.4% of the positions with consensus at 95% of positions, suggesting that the mutations defined in the maize background would have a similar effect in EPSPS from other crop species. To identify which positions to mutate in the EPSPS of the desired species, we used vNTI to align native maize EPSPS and optimized variants with the native sequences from the target species. ChloroP Prediction Server (ChloroP, a neural network-based method for predicting chloroplast transit peptides and their cleavage sites, Emanuelsson et al. Protein Sci. 8(5): 978-84, 1999) was used to approximate the amino terminus of the mature proteins. Nucleotide sequences were optimized for expression in E. coli and synthesized commercially. The synthetic genes were cloned into pHD2114 and expressed, purified and analyzed. In most but not all cases, the mutational combination defined in maize endowed a high level of fitness (k_{gly}) in the alternative plant EPSPS (Table 11).

TABLE 11
Efficiency of translation of mutations from maize
variant D2-124 to EPSPS from other crop species

| Enzyme source | Accession | k_{gly} | SE |
|---|---|---|---|
| Zea mays; corn | | 175 | 2.5 |
| Oryza sativa; rice | AF413082.1 | 174 | 3.1 |
| Sorghum bicolor; sorghum | XM_002436379.2 | 172 | 8.5 |
| Helianthus annuus; sunflower | XM_022161807.1 | 155 | 7.7 |
| Vitis vinifera; grapevine | NC_012021.3 | 144 | 10.6 |
| Gossypium hirsutum; cotton | UniProt A7Y7Y2 | 143 | 8.4 |
| Manihot esculenta; cassava | XM_021758443.1 | 133 | 6.3 |
| Glycine max; soybean | XM_003516991.3 | 114 | 4.1 |
| Triticum aestivum; wheat | ACH72672.1 | 102 | 6.1 |

The 18 mutations present in maize variant D2-124 were mapped onto the amino acid sequence of the predicted mature form of EPSPS from the species shown. Proteins were expressed and purified as described in Methods.

Fitness is judged by the value of k_{gly}, the enzyme turnover (min⁻¹) at 30 μM PEP and S3P, 1 mM glyphosate.

The mapped variants in some species (Sorghum, Helianthus, Oryza, Gossypium) had k_{gly} values almost as high as Zm D2-124.

1. A polynucleotide encoding a plant EPSP synthase (EPSPS) polypeptide, wherein the plant EPSPS polypeptide comprises G102A and at least one or more amino acid mutations selected from the group consisting of A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO: 1 and wherein the plant EPSPS polypeptide comprises an amino acid sequence that is at least 90% identical to SEQ ID NO: 2.

2. The polynucleotide of claim 1, wherein the polynucleotide encodes a plant EPSPS polypeptide that comprises G102A and at least two or more amino acid mutations.

3. The polynucleotide of claim 1, wherein the polynucleotide encodes a plant EPSPS polypeptide variant designated Zm D2-3P 124 (D2-124) that comprises A2R, G3K, A4W, H54G, A69H, K71E, K84R, L98C, I208L, K224R, K243E, V333A, A354G, E391P, D402G, and A416G.

4. The polynucleotide of claim 1, wherein the polynucleotide encodes a plant EPSPS polypeptide variant designated Zm D2c-A5 that comprises A2S, G3K, A4W, H54G, A69H, K71E, K84R, L98C, N162R, I208L, K224R, K243E, M293L, E302P, V333A, A354G, E391P, D402G, and A416G.

5. (canceled)

6. (canceled)

7. The polynucleotide of claim 1, wherein the polynucleotide encodes a plant EPSPS polypeptide variant designated Zm D2 that comprises A2R, A4W, A69H, K84R, L98C, I208L, K243E, V333A, E391P, and D402G.

8. (canceled)

9. (canceled)

10. (canceled)

11. The polynucleotide of claim 1, wherein the polynucleotide encodes the plant EPSPS polypeptide set forth in one of SEQ ID NOS: 3-12.

12. A recombinant DNA construct comprising the polynucleotide of claim 1.

13. A plant cell comprising the polynucleotide of claim 1.

14. The plant cell of claim 13, wherein said plant cell is a maize cell.

15. A plant comprising in its genome the polynucleotide of claim 1.

16. The plant of claim 15, wherein said plant is maize.

17. A seed comprising in its genome the polynucleotide of any one of claim 1.

18. The seed of claim 17, wherein said seed is maize seed.

19. A method of generating a glyphosate tolerant plant, the method comprising:
a) expressing in a regenerable plant cell a recombinant DNA construct comprising a polynucleotide operably linked to at least one regulatory sequence, wherein the polynucleotide encodes a plant EPSP synthase (EPSPS) polypeptide that comprises G102A and at least one amino acid mutation selected from the group consisting of: A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1 and wherein the plant EPSPS polypeptide comprises a sequence that is at least 90% identical to SEQ ID NO:2; and
b) generating the glyphosate tolerant plant, wherein the glyphosate tolerant plant comprises in its genome the recombinant DNA construct.

20. The method of claim 19, wherein said method comprises expressing in a plant cell a recombinant DNA construct comprising a polynucleotide encoding a plant EPSPS polypeptide comprising G102A and at least two amino acid mutations.

21. The method of claim 19, wherein said method comprises expressing in a plant cell a recombinant DNA construct comprising a polynucleotide encoding a plant EPSPS polypeptide comprising A2R, G3K, A4W, H54G, A69H, K71E, K84R, L98C, I208L, K224R, K243E, V333A, A354G, E391P, D402G, and A416G.

22. The method of claim 19, wherein said method comprises expressing in a plant cell a recombinant DNA construct comprising a polynucleotide encoding a plant EPSPS polypeptide comprising H54G, L98C, R216V, E226Y, K297A, V333A, T361S, D402G, and R429A.

23. The method of claim 19, wherein said method comprises expressing in a plant cell a recombinant DNA construct comprising a polynucleotide encoding a plant EPSPS polypeptide comprising L98C, T361S, and D402G.

24. The method of claim 19, wherein said method comprises expressing in a plant cell a recombinant DNA construct comprising a polynucleotide encoding a plant EPSPS polypeptide having the amino acid sequence set forth in one of SEQ ID NOS: 3-12 and 45-59.

25. A method of generating a glyphosate tolerant plant, said method comprising:
a) modifying an endogenous plant EPSP synthase (EPSPS) gene in a plant cell to encode a glyphosate tolerant EPSPS protein that comprises G102A and at least one amino acid mutation selected from the group consisting of A2R, A2S, G3K, A4W, S38A, H54L, H54G, A69H, K71E, K84R, E92G, L98C, N162R, I208L, R216V, K224R, E226Y, K243L, K243E, M293L, K297A, E302P, V333A, A354G, T361S, R368C, E391P, R429A, D402G, and A416G, wherein each amino acid mutation position corresponds to the amino acid position set forth in SEQ ID NO:1, and wherein the endogenous plant EPSPS gene encodes a polypeptide comprising a sequence that is at least 90% identical to SEQ ID NO:2; and
b) growing a plant from said plant cell, wherein said plant is tolerant to glyphosate.

26. The method of claim 25, wherein said modified endogenous plant EPSPS gene encodes a glyphosate tolerant EPSPS protein with at least two amino acid mutations selected.

27. The method of claim 25, wherein said modified endogenous plant EPSPS gene encodes a glyphosate tolerant EPSPS protein that comprises A2R, G3K, A4W, H54G, A69H, K71E, K84R, L98C, I208L, K224R, K243E, V333A, A354G, E391P, D402G, and A416G.

28. The method of claim 25, wherein said modified endogenous plant EPSPS gene encodes a glyphosate tolerant EPSPS protein that comprises H54G, L98C, R216V, E226Y, K297A, V333A, T361S, D402G, and R429A.

29. The method of claim 18, wherein said modified endogenous plant EPSPS gene encodes a glyphosate tolerant EPSPS protein that comprises L98C, T361S, and D402G.

30. (canceled)

31. The method of any one of claims 25-30, wherein the endogenous plant EPSPS gene has been modified by a CRISPR/Cas guide RNA-mediated system.

32-69. (canceled)
